(19) United States
(12) Patent Application Publication
(10) Pub. No.: US 2025/0085272 A1
(43) Pub. Date: Mar. 13, 2025

(54) METHODS AND SYSTEMS FOR MODULATING AND MODELING AGING AND NEURODEGENERATION DISEASES

(71) Applicant: MEMORIAL SLOAN-KETTERING CANCER CENTER, New York, NY (US)

(72) Inventors: Nathalie SAURAT, Long Island City, NY (US); Lorenz STUDER, New York, NY (US)

(73) Assignee: MEMORIAL SLOAN-KETTERING CANCER CENTER, New York, NY (US)

(21) Appl. No.: 18/960,311

(22) Filed: Nov. 26, 2024

Related U.S. Application Data
(63) Continuation of application No. PCT/US2023/023722, filed on May 26, 2023.
(60) Provisional application No. 63/346,182, filed on May 26, 2022.

(51) Int. Cl. G01N 33/50; C12N 5/0793

(52) U.S. Cl. CPC G01N 33/5058; C12N 5/0619; G01N 33/5023; C12N 2501/998; C12N 2503/02

(57) ABSTRACT

The present disclosure relates to methods for modulating cellular aging and/or progression of neurodegenerative diseases (e.g., AD). In certain embodiments, the methods induce cellular aging. In certain embodiments, the methods promote progression of neurodegenerative diseases (e.g., AD). The present disclosure also relates to methods and systems for modeling aging related neurodegenerative diseases (e.g., AD) in vitro. In certain embodiments, the methods disclosed herein comprise inhibiting protein neddylation pathway. In certain embodiments, inhibiting protein neddylation pathway comprises knocking out or knocking down genes (e.g., UBA3, NAE1) that regulate protein neddylation pathway. In certain embodiments, inhibiting protein neddylation pathway comprises administration a neddylation inhibitor (e.g., MLN4924) to cells.

### PRIORITY

This patent application is a continuation of International Patent Application No. PCT/US2023/023722, filed May 26, 2023, which claims priority to U.S. provisional application 63/346,182 filed May 26, 2022, the contents of each of which is incorporated herein by reference in its entirety, and to each of which priority is claimed.

### GRANT INFORMATION

The present disclosure was made with government support under Grant Nos. 1R01AG056298 and 1R01AG054720 awarded by the National Institute of Health. The government has certain rights in the disclosure.

### SEQUENCE LISTING

A Sequence Listing conforming to the rules of WIPO Standard ST.26 is hereby incorporated by reference. Said Sequence Listing has been filed as an electronic document via PatentCenter in ASCII format encoded as XML. The electronic document, created on Nov. 26, 2024, is entitled “0727341451_SL.xml”, and is 54,838 bytes in size.

### INTRODUCTION

The present disclosure relates to methods for modulating cellular aging and/or progression of neurodegenerative diseases (e.g., Alzheimer's disease). The present disclosure also relates to methods and systems for modeling aging related neurodegenerative diseases (e.g., Alzheimer's disease) in vitro.

### BACKGROUND

Alzheimer's disease (AD) has been intensely studied since mutations in APP and PSEN were linked to AD over 25 years ago. Despite the enormous amount of research on this topic, there has been limited success in translating findings into therapies that impact disease outcomes in AD patients. The risk of developing AD increases markedly with age raising the question of how the aging process may contribute to the development of AD. Answering this question has been challenging as AD is a largely human-specific disease, and human aging occur over periods of several decades, a process difficult to capture in any experimental system. Therefore, there is great need to develop a platform that allows controlled manipulations to either age or rejuvenate human brain cells on demand.

### SUMMARY OF THE INVENTION

The present disclosure relates to methods for modulating cellular aging and/or progression of neurodegenerative diseases (e.g., AD). The present disclosure also relates to methods and systems for modeling aging related neurodegenerative diseases (e.g., AD) in vitro.

In certain embodiments, the present disclosure provides methods of preparing an in vitro model of neurodegenerative disease comprising modulating protein neddylation in a population of neurons, wherein the population of neurons exhibit genetic mutation of at least one gene that is associated with neurodegenerative disease. The method of claim 1, wherein the neurodegenerative disease is Alzheimer's disease, Parkinson's disease, Amyotrophic Lateral Sclerosis (ALS), or Huntington's disease. In certain embodiments, modulating protein neddylation comprises exposing the population of neurons to a compound that modulates protein neddylation. In certain embodiments, the at least one compound that modulates protein neddylation is selected from the group consisting of MLN4924, TAS4464, CSN5i-3, ZM223, NACM-OPT, Keap1-Nrf2-IN-4, WS-383, VII-31, derivatives thereof, and combinations thereof. In certain embodiments, modulating protein neddylation comprises modifying expression of at least one gene which regulates protein neddylation pathways. In certain embodiments, the at least one gene which regulates protein neddylation pathways is selected from the group consisting of UBA3, NAE1, and combinations thereof. In certain embodiments, the mutation of at least one gene that is associated with neurodegenerative disease results in increased production of amyloid-beta peptide. In certain embodiments, the mutation of at least one gene that is associated with neurodegenerative disease results in a change in the ratio of Aβ40 to Aβ42 peptide.

In certain embodiments, the at least one gene that is associated with neurodegenerative disease comprises the Amyloid-beta precursor protein (APP) gene. In certain embodiments, the mutation of the APP gene comprises K595N/M596L. In certain embodiments, the at least one gene that is associated with neurodegenerative disease comprises the presenilin-1 (PSEN) gene. In certain embodiments, the mutation of the PSEN gene comprises M146V. In certain embodiments, the mutation of at least one gene that is associated with neurodegenerative disease results in increased production of N-ethylmaleimide sensitive factor (NSF) aggregates. In certain embodiments, the at least one gene that is associated with neurodegenerative disease comprises the (LRRK2) gene. In certain embodiments, the mutation of the LRRK2 gene comprises G2019S.

In certain embodiments, the neurons are obtained from in vitro differentiation of stem cells. In certain embodiments, the stem cells are human stem cells. In certain embodiments, the human stem cells are selected from the group consisting of human embryonic stem cells, human induced pluripotent stem cells, human parthenogenetic stem cells, human primordial germ cell-like pluripotent stem cells, human epiblast stem cells, human F-class pluripotent stem cells, and combinations thereof. In certain embodiments, the neurons are cortical neurons.

In certain embodiments, the present disclosure provides methods of identifying genes associated with cellular aging and/or progression of neurodegenerative disease comprising: obtaining a first population of neurons; obtaining a second population of neurons, and modifying expression of a test gene in the second population of neurons; measuring functional activity of the second population of neurons relative to the first population of neurons; wherein the first population of neurons and the second population of neurons exhibit genetic mutation of at least one gene that is associated with neurodegenerative disease; and wherein a difference in the functional activity between the first population of neurons and the second population of neurons indicates that the test gene is associated with cellular aging and/or progression of neurodegenerative disease. In certain embodiments, the functional activity is cell viability. In certain embodiments, the neurodegenerative disease is Alzheimer's disease, Parkinson's disease, Amyotrophic Lateral Sclerosis (ALS), or Huntington's disease. In certain embodiments, modifying expression of the test gene modulates protein neddylation in the second population of neurons.

In certain embodiments, the mutation of at least one gene that is associated with neurodegenerative disease results in increased production of amyloid-beta peptide. In certain embodiments, the mutation of at least one gene that is associated with neurodegenerative disease results in a change in the ratio of Aβ40 to Aβ42 peptide. In certain embodiments, the at least one gene that is associated with neurodegenerative disease comprises the Amyloid-beta precursor protein (APP) gene. In certain embodiments, the mutation of the APP gene comprises K595N/M596L. In certain embodiments, the at least one gene that is associated with neurodegenerative disease comprises the presenilin-1 (PSEN) gene. In certain embodiments, the mutation of the PSEN gene comprises M146V. In certain embodiments, the mutation of at least one gene that is associated with neurodegenerative disease results in increased production of N-ethylmaleimide sensitive factor (NSF) aggregates. In certain embodiments, the at least one gene that is associated with neurodegenerative disease comprises the (LRRK2) gene. In certain embodiments, the mutation of the LRRK2 gene comprises G2019S.

In certain embodiments, the neurons are obtained from in vitro differentiation of stem cells. In certain embodiments, the stem cells are human stem cells. In certain embodiments, the human stem cells are selected from the group consisting of human embryonic stem cells, human induced pluripotent stem cells, human parthenogenetic stem cells, human primordial germ cell-like pluripotent stem cells, human epiblast stem cells, human F-class pluripotent stem cells, and combinations thereof. In certain embodiments, the neurons are cortical neurons.

In certain embodiments, the present disclosure provides compositions for identifying genes associated with cellular aging and/or progression of neurodegenerative disease comprising a population of neurons, wherein the population of neurons exhibit genetic mutation at a test gene, wherein the population of neurons exhibit genetic mutation of at least one gene that is associated with neurodegenerative disease. In certain embodiments, the neurodegenerative disease is Alzheimer's disease, Parkinson's disease, Amyotrophic Lateral Sclerosis (ALS), or Huntington's disease. In certain embodiments, the genetic mutation at the test gene modulates protein neddylation.

In certain embodiments, the mutation of at least one gene that is associated with neurodegenerative disease results in increased production of amyloid-beta peptide. In certain embodiments, the mutation of at least one gene that is associated with neurodegenerative disease results in a change in the ratio of Aβ40 to Aβ42 peptide. In certain embodiments, the at least one gene that is associated with neurodegenerative disease comprises the Amyloid-beta precursor protein (APP) gene. In certain embodiments, the mutation of the APP gene comprises K595N/M596L In certain embodiments, the at least one gene that is associated with neurodegenerative disease comprises the presenilin-1 (PSEN) gene. In certain embodiments, the mutation of the PSEN gene comprises M146V. In certain embodiments, the mutation of at least one gene that is associated with neurodegenerative disease results in increased production of N-ethylmaleimide sensitive factor (NSF) aggregates. In certain embodiments, the at least one gene that is associated with neurodegenerative disease comprises the (LRRK2) gene. In certain embodiments, the mutation of the LRRK2 gene comprises G2019S.

In certain embodiments, the neurons are obtained from in vitro differentiation of stem cells. In certain embodiments, the stem cells are human stem cells. In certain embodiments, the human stem cells are selected from the group consisting of human embryonic stem cells, human induced pluripotent stem cells, human parthenogenetic stem cells, human primordial germ cell-like pluripotent stem cells, human epiblast stem cells, human F-class pluripotent stem cells, and combinations thereof. In certain embodiments, the neurons are cortical neurons.

### BRIEF DESCRIPTION OF THE DRAWINGS

FIGS. 1A-1N show the genome-wide Crispr Cas9 screen in an isogenic stem-cell model of fAD identifying genotype specific regulators of neuron viability. FIG. 1A shows schema outlining cell line engineering for this study. H9 PSCs were sequentially engineered to generate isogenic cell lines for this study. Dox-inducible Cas9 was knocked into the AAVS1 locus followed by homozygous knock-in of the APP Swedish mutation. FIG. 2B shows that sequencing confirms correct knock-in of the APP Swedish mutation. FIG. 3C shows qPCR quantification of Cas9 induction after 48 hour treatment with doxycycline (mean±s.d., n=5 independent passages for stem cells and n=5 independent differentiations for neurons). FIG. 1D shows quantification of neurons and cycling contaminants in cultures of terminally differentiated neurons (mean±s.d., n=3 independent differentiations). FIG. 1E shows quantification of amyloid peptide production in cortical neurons 35 days after neural induction. Total aβ peptide represents the sum of aβ38, aβ40 and aβ42 in cell culture supernatant (mean±s.d., n=3 independent inductions, unpaired two tailed t-test). FIG. 1F shows Western blotting for phospho TauS202/T205 (AT8) and Total Tau (T-Tau) in cortical neurons harvested 65 days after neuron induction. FIG. 1G shows quantification of western blots in FIG. 1F (mean±s.d., n=3 independent inductions). FIG. 1H shows Presto blue viability assay performed on cortical neurons 65 days after neuron induction (mean±s.d., n=6 independent inductions). FIG. 1I shows schema outlining the workflow and design of the whole genome CRISPR/Cas9 screen. FIG. 1J shows interpretation guide for the whole genome CRISPR/Cas9 screen. FIG. 1K shows scatter plot of beta scores for each gene in wild-type and APP^{swe/swe} neurons. Genes reaching significance (p<0.05 and FDR<0.3) are colored according to categories indicated in FIG. 2B. FIG. 1L shows KEGG pathway analysis of guideRNAs whose loss of function increases cell number in both genotypes at the screen endpoint (Survival or proliferation genes). FIG. 1M shows KEGG pathway analysis of guideRNAs whose loss of function decreases cell number in both genotypes at the screen endpoint (essential genes). FIG. 1N shows KEGG pathway analysis of guideRNAs whose loss of function significantly decreases cell number in only the APP^{swe/swe} neurons at the screen endpoint (AD specific viability genes).

FIGS. 2A-2G show that experimentally validated hit genes do not alter APP processing, but a subset are decreased during physiological aging. FIG. 2A shows outline of strategy used to rank genes for secondary validation. FIG. 2B shows secondary validation of top ranked ‘hit’ genes from the whole genome CRISPR Cas9 screen. Viability was assayed using the Presto viability assay and normalized to −Dox control for each guide/genotype combination (mean±s.d., n=5 independent inductions). P-values were calculated using the Student's t-test to compare the WT +Dox to the App^{swe/swe} +Dox conditions (*=P-value<0.05, **P-value<0.01). FIG. 2C shows schema outlining potential mechanism underlying AD-enhanced loss of viability. It was hypothesized that hits would either potentiate existing AD phenotypes or drive cellular age. FIG. 2D shows quantification of total (Aβ38, 40, 42) extracellular Aβ peptide production in DIV45 APP^{swe/swe} neurons after knockout of hits validated in FIG. 2B. AB measurements were scaled to total protein and presented relative to the −Dox control for each gene knockout. Data presented as mean±s.d., cells from 3 independent inductions except for TOMM22 where Aβ38 was below the detection threshold in one of the +Dox replicates). FIG. 2E shows ratio of Aβ40 to Aβ42 peptide in cell culture in DIV 45 APP^{swe/swe} neurons normalized to the Aβ40:Aβ42 ratio of the-Dox control for each gene knockout. (mean±s.d., cells from 3 independent inductions). FIG. 2F shows quantification of RNA expression for the validated hit genes in young (12-14 years) and old (70-91 years) human cortex. Data from Cornacchia et al. (in preparation). (mean±s.d., n=9, unpaired two tailed t-test). FIG. 2G shows quantification of bulk RNA expression for the validated hit genes in young (1-6 months of age) and old (21-27 months of age) mouse brain. Data from the Tabula Muris Senis consortium⁷ (mean±s.d., n=18 (young) and n=13 (old), unpaired two tailed t-test).

FIGS. 3A-3E show that inhibiting neddylation induces cellular hallmarks of age in AD-PSC neurons. FIG. 3A shows outline of hallmarks of age assayed for in this study and the expected change in aged cells. FIG. 3B shows DIV APP^{swe/swe} cortical neurons treated with 1 μM of MLN4924 for 7 days. pATM puncta normalized to the number neurons per field (MAP2+). Bleomycin was used as a positive control. Graph shows the median of n=3 independent differentiations. P-values calculated using unpaired two tailed student's t-test. FIGS. 3C-3E show DIV 50 APP^{swe/swe} cortical neurons were treated with 1 μM of MLN4924 for 10 days before assaying for aging hallmarks: loss of proteostasis (FIG. 3C), loss of heterochromatin (FIG. 3D) and increased cellular senescence (FIG. 3E) by flow cytometry. Median intensity for each condition shown on plot.

FIGS. 4A-4G show that inhibiting neddylation results in AD-specific changes in phosphorylated Tau. FIG. 4A shows immunofluorescence at DIV 50 for total Tau and pTau(S235) in UBA3 knock out neurons. Yellow arrows indicate examples accumulations of pTau(S235) in neurites and white arrows indicate pTau(S235) accumulation in the cell body. These accumulations are examples of pTau(235)^{bright} immunoreactivity. Scale bar 100 μM. FIG. 4B shows quantification of pTau(S235) or pTau(235)^{bright} normalized to total Tau. P-values calculated using unpaired two-tailed t-test (mean, n=3 independent differentiations). FIGS. 4C-4F show western blotting for pTau(S202/T205) and total Tau in neurons treated 1 μM MLN4924 from DIV50-DIV60. Western blotting (FIG. 4C) and quantification (FIG. 4D) for wild-type neurons. (Paired student's t-test (NS p>0.05); n=3 independent differentiations). Western blotting (FIG. 4E) and quantification (FIG. 4F) for App^{swe/swe} neurons (Paired student's t-test; n=4 independent differentiations). FIG. 4G shows summary of findings. Blocking neddylation and inducing cellular age potentiates late onset AD phenotypes including an AD specific increase in pTau and an AD enhanced loss neuronal loss (right panels). These phenotypes are not seen in non-aged neurons (left panels).

FIGS. 5A-5H shows characterization and validation of iCas9 pluripotent stem cell lines. FIG. 5A shows karyotype analysis of isogenic stem cell lines generated for this study. FIG. 5B shows immunofluorescence showing induction of Cas9 in PSCs after 48 h of doxycycline treatment. Scale bar 50 μm. FIGS. 5C-5D show Western blotting for Cas9 induction in PSCs (FIG. 5C) or DIV 22 neurons (FIG. 5D) after 48 hour treatment with doxycycline. FIG. 5E shows Western blot showing loss of Cas9 protein after doxycycline is withdrawn from cortical neuron cultures. FIG. 5F shows Western blot for Cas9 after 48 h of doxycycline addition in DIV 20 and DIV 27 cultures showing that the inducible Cas9 construct silences soon after terminal differentiation to postmitotic neurons. FIG. 5G shows schema outlining the TD-Tomato assay used to functionally validate inducible Cas9 platform. This experimental set up mirrors the one used for the WGS. FIG. 5H shows immunofluorescence for TD-tomato 10 days after adding doxycycline to cortical cultures as outlined in FIG. 5G. Matched brightfield images are inset; scale bars 100 82 m.

FIGS. 6A-6D show overview and quality control of directed differentiation platform used for this study. FIG. 6A shows outline of the cortical neuron differentiation protocol used to generate neurons for this study. FIG. 6B shows qPCR quantification of OCT4, PAX6, FOXG1 and TUBB3 expression during cortical differentiation (mean±s.d., n=3 independent differentiations). FIG. 6C shows immunofluorescence for PAX6 after cortical differentiation (DIV 20). Scale bars 100 μm. FIG. 6D shows immunofluorescence for FOXG1/MAP2 in terminally differentiated cortical neurons (DIV 30). Scale bars 100 μm.

FIGS. 7A-7E show extended characterization of WGS. FIG. 7A shows outline of the Brunello sgRNA knockout library and cell numbers needed used for this screen. The library contains 4 guides per gene with a total of 76,441 guide RNAs. To maintain 1000× representation >76.4 million cells for each condition were maintained when culturing the stem cells and throughout the differentiation. Stem cells were transduced at 0.3 MOI to ensure single gRNA insertion per cell. FIG. 7B shows number of significantly enriched or depleted genes (p<0.05) in the +Dox sample relative to either the T=0 (Day 20) control or the endpoint −Dox control. FIG. 7C shows comparison of guide RNA representation across all conditions. FIG. 7D shows scatter plot of beta scores for each gene in wild-type and APP^{swe/swe} neurons with T=0 samples used for normalization. Genes reaching significance (p<0.05 and FDR <0.3) are colored according to categories indicated in FIG. 2B (green=essential, blue=‘hits’, yellow=proliferation/survival). Outlier genes are also indicated on the plot. FIG. 7E shows scatter plot of beta scores for each gene in wild-type and APP^{swe/swe} neurons with endpoint control samples used for normalization. Genes reaching significance (p<0.05 and FDR<0.3) are colored according to categories indicated in FIG. 2B (green=essential, blue=‘hits’, yellow=proliferation/survival).

FIGS. 8A-8D show transduction of neural precursors at DIV20 does not affect efficiency of gene knockout. FIG. 8A shows schema outlining the guideRNA transduction and selection protocol used for secondary screening of hit genes. FIG. 8B shows PSCs were transduced with control guideRNAs (non-targeting; TOMM22) and differentiated using the same platform as the WGS. Viability was measured 10 days after gene knockout at DIV30 (mean±s.d., n=3 independent differentiations). FIG. 8C shows viability 10 days after control gene knockout using the transduction and selection platform outlined in FIG. 8A and used for the secondary validation experiments (mean±s.d., n=3 independent differentiations). FIG. 8D shows example brightfield images 10 days after knockout of positive control gene (TOMM22).

FIGS. 9A-9G show validation of top ranked essential and proliferation genes from the WGS. FIG. 9A shows outline of strategy used to rank essential and proliferation genes for secondary validation. FIG. 9B shows ranked genes indicating the top essential genes (PPIAL4E and NUDT6) and the top proliferation/survival gene (SUFU). FIGS. 9C-9E show validation of top ranked essential genes, PPIAL4E (FIG. 9C) and NUDT6 (FIG. 9D). Viability was assayed at DIV 60 using the Presto viability assay and normalized to the −Dox control (mean±s.d., n=3 independent differentiations). FIG. 9E shows validation of top ranked proliferation/survival gene. Viability was assayed at DIV 60 using the Presto viability assay and normalized to the-Dox control (mean±s.d., n=3 independent differentiations). FIG. 9F-9G show immunofluorescence for PAX6, Ki67 and MAP2 (FIG. 9F) or COL1A1 (FIG. 9G) in wild-type SUFU knockout neurons or unedited controls (DIV 30).

FIG. 10 shows secondary validation of WGS. Secondary validation of hit genes from the secondary screen. Presto blue viability assay was used to measure cell viability at DIV60, and viability was normalized to the −Dox control (mean±s.d., n=5 independent differentiations, unpaired two-sided t-test).

FIGS. 11A-11B show source data for normalization of Aβ measurements. FIG. 11A shows quantification of total (Aβ38, 40, 42) extracellular Aβ peptide production prior to normalization to account for neuron number. Culture media was harvested from DIV45 APP^{swe/swe} neurons after knockout of validated hit gene. Data presented as mean±s.d., cells from 3 independent inductions except for TOMM22 where Aβ38 was below the detection threshold in one of the +Dox replicates. FIG. 11B shows quantification of total protein in DIV45 APP^{swe/swe} neurons after knockout of validated hit gene. Total protein was normalized to the −Dox sample. (mean±s.d., cells from 3 independent inductions and transductions).

FIGS. 12A-12I show that inhibiting the neddylation pathway results in an AD-enhanced loss of viability. FIG. 12A shows beta scores from whole genome Crispr screen for UBA3 and NAE1 in WT and APP^{swe/swe} neurons. T=0 and endpoint refer to screen analysis with the DIV20 samples or the DIV65 (−Dox) as the control samples, respectively. Significance refers to P-value from the screen. FIGS. 12B-12C show validation of sgRNA for NAE1 (FIG. 12B) or UBA3 (FIG. 12C) knockout. Gene knockout was performed in DIV20 neurons and cells were harvested for western blotting 10 days later. FIGS. 12D-12E show Western blotting (FIG. 12D) and quantification (FIG. 12E) of UBA3 protein reduction in APP^{swe/swe} neurons transduced with sgRNAs targeting UBA3. Gene knockout was performed in DIV20 neurons and cells were harvested for western blotting at DIV 40 (mean±s.d., cells from 4 independent inductions and transductions). P-values were calculated using the Student's t-test. FIG. 12F shows Western blotting to confirm 1 μM MLN4924 blocks conjugation of NEDD8 to target proteins in cortical neurons after 3 hours or 7 days of treatment. FIG. 12G shows CCK8 viability assay in UBA3 knockout neurons. Gene knockout was performed at DIV20, and viability was assayed at DIV60 (mean±s.d., n=5 independent inductions/experiments). FIG. 12H shows Presto blue viability assay in NAE1 knockout neurons. Gene knockout was performed at DIV20, and viability was assayed at DIV60 (mean±s.d., n=5 independent inductions/experiments). FIG. 12I shows Presto blue viability assay in MLN4924 treated neurons. 1 μM of MLN4924 was added to the culture media from DIV30 and Presto blue viability assays were performed at DIV60 (mean±s.d., n=4 independent inductions/experiments). P-values were calculated using the Student's t-test.

FIGS. 13A-13G show that Western blotting confirms loss of proteostasis and increased cellular senescence in neurons treated with a neddylation inhibitor. FIG. 13A shows example immunofluorescence image showing pATM induction in APP^{swe/swe} neurons treated with (1 μM) MLN4924 for 7 days. Scale bars 100 μm. FIG. 13B shows brightfield images showing induction of showing β-gal positive APP^{swe/swe} neurons after 10 days treatment with 1 μM MLN4924. Scale bars 100 μm. FIG. 13C shows Western blotting for p21 and GAPDH loading control in APP^{swe/swe} neurons treated with 1 μM MLN4924 from DIV50-DIV60 (Paired t-test; n=4 independent differentiations). FIG. 13D shows Western blotting for LAMNB1 and GAPDH loading control in APP^{swe/swe} neurons treated with 1 μM MLN4924 from DIV50-DIV60 (Paired t-test; n=4 independent differentiations). FIG. 13E shows Western blotting for BAG1 and GAPDH loading control in APP^{swe/swe} neurons treated with 1 μM MLN4924 from DIV50-DIV60. FIG. 13F shows Western blotting for BAG3 and GAPDH loading control in APP^{swe/swe} neurons treated with 1 μM MLN4924 from DIV50-DIV60. FIG. 13G shows quantification of western blots in FIGS. 13E-13F (Paired t-test; n=4 independent inductions).

FIGS. 14A-14D show the FACS gating scheme. FIG. 14A shows cells gated from debris based on Forward Scatter Area (FSC-A) and Side Scatter Area (SSC-A). FIG. 14B shows single cells gated from doublets using Side Scatter Width (SSC-W) and Side Scatter Area (SSC-A). FIG. 14C shows single cells further gated from doublets using Forward Scatter Width (FSC-W) and Forward Scatter Area (FSC-A). FIG. 14D shows live cells isolated from dead cells and/or debris using Zombie-UV fixed viability day and Autofluorescence (AF).

FIG. 15 shows Alzheimer's disease deaths per 100,000 per year. Edited from: The Milbank Quarterly, Vol. 80, No. 1, 2002 from 1997 U.S. Vital Statistics.

FIG. 16 describes the gain of function screen. Identify genes whose loss of function leads to death of APP^{swe/swe} neurons.

FIGS. 17A-17C show validation for genome engineering. FIG. 17A shows outline of genome engineering steps. FIG. 17B shows that Cas9 can be induced to equivalent levels in PSCs and differentiated cells in both cell lines. FIG. 17C shows confirmation of APP APP^{swe/swe} mutation knock-in.

FIGS. 18A-18E show validation for disease modelling. FIG. 18A shows that directed differentiation to cortical neurons generates postmitotic neurons with <1% cycling cells. FIG. 18B shows summary of AD phenotypes present in vitro. FIG. 18C shows that APP^{swe/swe} mutation results in increased production of extracellular Aβ. FIG. 18D shows quantification of western blots shows no difference in total Tau or pTau between genotypes. FIG. 18E shows there is no difference in neuronal viability between genotypes.

FIGS. 19A-19E illustrate the whole genome Crispr/Cas9 screen. FIG. 19A shows the outline of WGS. FIG. 19B shows the interpretation guide for WGS. FIG. 19C shows results of WGS indicating hits in each of the categories outlined in FIG. 19B. FIG. 19D shows KEGG pathway analysis for essential genes. FIG. 19E shows KEGG pathway analysis for genes showing an AD enhanced loss of viability.

FIGS. 20A-20B show that neddylation inhibition leads to AD-enhanced loss of viability. FIG. 20A shows that both E1 ligases for neddylation (NAE1 and UBA3) were hits in the WGS. FIG. 20B shows the AD-enhanced vulnerability in response to neddylation loss of function shown in orthogonal independent viability assays.

FIGS. 21A-21C show that blocking neddylation does not impact APP processing. FIG. 21A shows potential mechanisms for AD-enhanced loss of viability. FIGS. 21B-21C show MSD ELISA based quantification of extracellular Aβ production which shows no change in APP processing in UBA3-KO. Aβ production is quantified based on total extracellular Aβ (FIG. 21B) or the ratio of Aβ40 to Aβ42 (FIG. 21C). NT=non-targeting, TOMM22=positive control for loss of viability.

FIG. 22A-22E show that UBA3 is reduced in the aged brain and blocking neddylation induces hallmarks of age. FIG. 22A-22B show that UBA3 is differentially expressed in old and young human (FIG. 22A) and mouse (FIG. 22B) cortex. FIG. 22C shows an overview of hallmarks of cellular age. FIGS. 22D-22E show that neddylation loss of function increased DNA damage (FIG. 22D) protein aggregation and senescence and decreased histone methylation (FIG. 22E).

FIGS. 23A-23E show that neddylation loss of function induces AD-specific changes in pTAU. FIGS. 23A shows that AD neurons treated with the neddylation inhibitor showed a decrease in major form of pTAU(AT8) and an increase in the high molecular weight form. FIG. 23B shows quantification of FIG. 23A. FIG. 23C shows that wild-type neurons did not show the change observed in FIGS. 23A-23B. FIGS. 23D shows that UBA3 KO leads to AD-specific increase in pTau(235) inclusions. FIG. 23E shows quantification of FIG. 23D.

FIG. 24 shows that genetics and age synergize to potentiate AD.

FIGS. 25A-25B show that inhibiting neddylation can be used when modelling other genetic forms of AD. FIG. 25A shows the production of neurons having the PSEN^{M146V/M146V} mutation. FIG. 25B shows that neurons with the PSEN^{M146V/M146V} mutation exhibited reduced viability following treatment with MLN4924.

FIGS. 26A-26C show that regulators of age identified in Alzheimer's Disease whole genome CRISPR screen can also be used to model Parkinson's Disease neurodegeneration in vitro. FIG. 26A shows the production of H9-Nurr1^{GFP} neurons having the LRRK2 G2019S mutation. FIG. 26B shows expression of Nurr1GFP and tyrosine hydroxylase (TH) with DAPI counterstain. FIG. 26C shows that neurons with the LRRK2 G2019S mutation exhibited reduced viability following treatment with MLN4924.

FIGS. 27A-27B show that inhibiting neddylation induces cellular age in both wild-type and disease backgrounds. FIG. 27A plots protein aggregation, senescence, and histone methylation for wild-type or APP^{swe/swe} neurons following treatment with MLN4924 or DMSO. FIG. 27B shows quantification of FIG. 27A.

FIGS. 28A-28B show additional hallmarks of aging. FIG. 28A shows that AD neurons exhibit decreased nuclear roundness, i.e., indication of nuclear lamina defects, following treatment with MLN4924. FIG. 28B shows that AD neurons exhibit increased nuclear area, i.e., indication of cellular senescence, following treatment with MLN4924.

### DETAILED DESCRIPTION

The present disclosure relates to methods for modulating cellular aging and/or progression of neurodegenerative diseases (e.g., AD). The present disclosure also relates to methods and systems for modeling aging related neurodegenerative diseases (e.g., AD) in vitro.

In certain embodiments, the present disclosure is based on the discovery that neddylation pathway regulates both cellular age and AD-neurodegeneration. Specifically, the present disclosure identifies that blocking neddylation increased cellular senescence, protein aggregation and DNA damage and decreased heterochromatin in cortical neurons. Blocking neddylation also led to an increase in high molecular weight phosphorylated Tau that was specific to neurons with the APP^{swe/swe} mutation. Finally, aged APP^{swe/swe} neurons also showed a greater loss of viability than wild-type neurons

The present disclosure provides a genome screening platform to identify physiologically relevant regulators of cellular age and AD-neurodegeneration. The present disclosure defines hit genes as those whose loss of function selectively compromises the viability of Alzheimer's disease but not control isogenic neurons. The present disclosure identifies that AD-enhanced loss of viability resulted from the synergistic action of the AD genetic susceptibility with a screen-induced age-related vulnerability. The present disclosure shows that experimentally validated hit genes selectively compromised the viability of Alzheimer's disease neurons over isogenic control neurons but did not impact APP processing. Consistent with the hypothesis that age-related vulnerability can synergize with genetic susceptibility, 4 of the 6 experimentally validated hits showed a significant decrease in expression in aged human and mouse primary brain tissue compared to matched young samples. While the present disclosure focuses on UBA3 for further characterization, NAE1, the other member of the heterodimeric E1 ligase for Nedd8 was also a hit in the screen, and chemical inhibition of the neddylation pathway similarly resulted in an AD enhanced loss of viability. Consistent with age-related phenotype, blocking neddylation triggered known hallmarks of age including cellular senescence, DNA damage, loss of proteostasis and a global reduction in heterochromatin. Finally, blocking neddylation also led to an AD-specific increase in high molecular weight phosphorylated Tau and resulted in phospho-Tau positive inclusions.

The present disclosure demonstrates how cellular age and disease genetics can synergize to trigger late-onset disease phenotypes. The present disclosure uses developmentally defined cortical neurons generated by directed differentiation (rather than transcription-factor based iNeurons) to perform a whole genome CRISPR screen. In addition, the present disclosure takes one of the major challenges of stem cell models of neurodegenerative disease—namely that late onset phenotypes like neuronal loss have been challenging to model in vitro due to their embryonic nature of hPSC-derived cells—and uses this as the basis for a phenotypic “gain-of-disease” screen. The presently disclosed aged AD-PSC model can be used in screening for drugs that can prevent disease progression and neuronal loss. Finally, the present disclosure also has broad implications for human disease modelling, as it highlights the importance of generating cells of the appropriate “age” in addition to the correct developmental lineage and cellular identity.

For purposes of clarity of disclosure and not by way of limitation, the detailed description is divided into the following subsections:
1. Definitions;
2. PSC-based Models of Neurodegenerative Diseases
3. Inhibiting Protein Neddylation Pathways
4. Identification of Genes Associated with Cellular Aging and/or Neurodegenerative Disease

### 1. Definitions

The terms used in this disclosure generally have their ordinary meanings in the art, within the context of this disclosure and in the specific context where each term is used. Certain terms are discussed below, or elsewhere in the specification, to provide additional guidance to the practitioner in describing the compositions and methods of the disclosure and how to make and use them.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

As used herein, the term “stem cell” refers to a cell with the ability to divide for indefinite periods in culture and to give rise to specialized cells. In certain embodiments, a stem cell can refer to an embryonic stem cell or an induced pluripotent stem cell (iPSC). A human stem cell refers to a stem cell that is derived from a human.

As used herein, the term “embryonic stem cell” refers to a primitive (undifferentiated) cell that is derived from preimplantation-stage embryo, capable of dividing without differentiating for a prolonged period in culture, and are known to develop into cells and tissues of the three primary germ layers. A human embryonic stem cell refers to an embryonic stem cell that is from a human. As used herein, the term “human embryonic stem cell” or “hESC” refers to a type of pluripotent stem cells derived from early stage human embryos, up to and including the blastocyst stage, that is capable of dividing without differentiating for a prolonged period in culture, and are known to develop into cells and tissues of the three primary germ layers.

As used herein, the term “embryonic stem cell line” refers to a population of embryonic stem cells which have been cultured under in vitro conditions that allow proliferation without differentiation for up to days, months to years. For example, “embryonic stem cell” can refers to a primitive (undifferentiated) cell that is derived from preimplantation-stage embryo, capable of dividing without differentiating for a prolonged period in culture, and are known to develop into cells and tissues of the three primary germ layers. A human embryonic stem cell refers to an embryonic stem cell that is from a human. As used herein, the term “human embryonic stem cell” or “hESC” refers to a type of pluripotent stem cells derived from early stage human embryos, up to and including the blastocyst stage, that is capable of dividing without differentiating for a prolonged period in culture, and are known to develop into cells and tissues of the three primary germ layers.

As used herein, the term “pluripotent” refers to an ability to develop into the three developmental germ layers of the organism including endoderm, mesoderm, and ectoderm.

As used herein, the term “induced pluripotent stem cell” or “iPSC” refers to a type of pluripotent stem cell, similar to an embryonic stem cell, formed by the introduction of certain embryonic genes (see, for example, Takahashi and Yamanaka Cell 126, 663-676 (2006), herein incorporated by reference) into a somatic cell.

As used herein, the term “somatic cell” refers to any cell in the body other than gametes (egg or sperm); sometimes referred to as “adult” cells.

As used herein, the term “somatic (adult) stem cell” refers to a relatively rare undifferentiated cell found in many organs and differentiated tissues with a limited capacity for both self-renewal (in the laboratory) and differentiation. Such cells vary in their differentiation capacity, but it is usually limited to cell types in the organ of origin.

As used herein, the term “proliferation” refers to an increase in cell number.

As used herein, the term “undifferentiated” refers to a cell that has not yet developed into a specialized cell type.

As used herein, the term “differentiation” refers to a process whereby an unspecialized embryonic cell acquires the features of a specialized cell such as a heart, liver, or muscle cell. Differentiation is controlled by the interaction of a cell's genes with the physical and chemical conditions outside the cell, usually through signaling pathways involving proteins embedded in the cell surface.

As used herein, the term “directed differentiation” refers to a manipulation of stem cell culture conditions to induce differentiation into a particular (for example, desired) cell type. In certain embodiments, the term “directed differentiation” in reference to a stem cell refers to the use of small molecules, growth factor proteins, and other growth conditions to promote the transition of a stem cell from the pluripotent state into a more mature or specialized cell fate (e.g., prefrontal cortex cells or neural crest cells, etc.).

As used herein, the term “inducing differentiation” in reference to a cell refers to changing the default cell type (genotype and/or phenotype) to a non-default cell type (genotype and/or phenotype). Thus, “inducing differentiation in a stem cell” refers to inducing the stem cell (e.g., human stem cell) to divide into progeny cells with characteristics that are different from the stem cell, such as genotype (e.g., change in gene expression as determined by genetic analysis such as a microarray) and/or phenotype (e.g., change in expression of a protein).

As used herein, the term “culture medium” refers to a liquid that covers cells in a culture vessel, such as a Petri plate, a multi-well plate, and the like, and contains nutrients to nourish and support the cells. Culture medium may also include growth factors added to produce desired changes in the cells.

An “effective amount” is an amount effective, at dosages and for periods of time necessary, that produces a desired effect, e.g., the desired therapeutic or prophylactic result.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

As used herein, the term “expressing” in relation to a gene or protein refers to making an mRNA or protein which can be observed using assays such as microarray assays, antibody staining assays, and the like.

As used herein, the term “marker” or “cell marker” refers to gene or protein that identifies a particular cell or cell type, e.g., prefrontal cortex cells or neural crest cells. A marker for a cell may not be limited to one marker, markers may refer to a “pattern” of markers such that a designated group of markers may identity a cell or cell type from another cell or cell type.

The terms “detection” or “detecting” include any means of detecting, including direct and indirect detection.

As used herein, the term “derived from” or “established from” or “differentiated from” when made in reference to any cell disclosed herein refers to a cell that was obtained from (e.g., isolated, purified, etc.) a parent cell in a cell line, tissue (such as a dissociated embryo, or fluids using any manipulation, such as, without limitation, single cell isolation, cultured in vitro, treatment and/or mutagenesis using for example proteins, chemicals, radiation, infection with virus, transfection with DNA sequences, such as with a morphogen, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure, and the like.

As used herein, the term “signaling” in reference to a “signal transduction protein” refers to a protein that is activated or otherwise affected by ligand binding to a membrane receptor protein or some other stimulus. Examples of signal transduction proteins include, but are not limited to, a SMAD, transforming growth factor beta (TGFB), Activin, Nodal, bone morphogenic (BMP) and NFIA proteins. For many cell surface receptors or internal receptor proteins, ligand-receptor interactions are not directly linked to the cell's response. The ligand activated receptor can first interact with other proteins inside the cell before the ultimate physiological effect of the ligand on the cell's behavior is produced. Often, the behavior of a chain of several interacting cell proteins is altered following receptor activation or inhibition. The entire set of cell changes induced by receptor activation is called a signal transduction mechanism or signaling pathway.

As used herein, the term “signals” refer to internal and external factors that control changes in cell structure and function. They can be chemical or physical in nature.

As used herein, the term “ligands” refers to molecules and proteins that bind to receptors, e.g., transforming growth factor-beta (TFGβ), Activin, Nodal, bone morphogenic proteins (BMPs), etc.

As used herein, the term “inhibitor” refers to a compound or molecule (e.g., small molecule, peptide, peptidomimetic, natural compound, siRNA, anti-sense nucleic acid, aptamer, or antibody) that interferes with (e.g., reduces, decreases, suppresses, eliminates, or blocks) the signaling function of the molecule or pathway. An inhibitor can be any compound or molecule that changes any activity of a named protein (signaling molecule, any molecule involved with the named signaling molecule, or a named associated molecule) (e.g., including, but not limited to, the signaling molecules described herein). Inhibitors are described in terms of competitive inhibition (binds to the active site in a manner as to exclude or reduce the binding of another known binding compound) and allosteric inhibition (binds to a protein in a manner to change the protein conformation in a manner which interferes with binding of a compound to that protein's active site) in addition to inhibition induced by binding to and affecting a molecule upstream from the named signaling molecule that in turn causes inhibition of the named molecule. An inhibitor can be a “direct inhibitor” that inhibits a signaling target or a signaling target pathway by actually contacting the signaling target.

“Activators”, as used herein, refer to compounds that increase, induce, stimulate, activate, facilitate, or enhance activation of a protein or molecule, or the signaling function of the protein, molecule or pathway.

As used herein, the term “derivative” refers to a chemical compound with a similar core structure.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” or “disorder” refers to any condition or disorder that damages or interferes with the normal function of a cell, tissue, or organ.

As used herein, the term “treating” or “treatment” refers to clinical intervention in an attempt to alter the disease course of the individual or cell being treated, and can be performed cither for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

The term “differentiation day” as used herein, refers to a time line having twenty-four-hour intervals (i.e., days) after a stem cell culture is contacted by differentiation molecules. For example, such molecules may include, but are not limited to, SMAD inhibitor molecules, BMP inhibitor molecules, WNT inhibitor molecules and BMP molecules. The day of contacting the culture with the molecules is referred to as differentiation day 1. For example, differentiation day 2 represents anytime between twenty-four and forty-eight hours after the stem cell culture had been contacted by a differentiation molecule.

As used herein, the term “gene” refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions as well as regulatory regions and can include 5′ and 3′ ends.

The term “multi-gene disorder” as used herein, refers to a disorder that results from the presence of mutations in two or more genes. In certain embodiments, patients having the same multi-gene disorder can harbor different single-gene mutations. In certain embodiments, a single patient having the multi-gene disorder can harbor mutations in multiple genes, and different patients having multi-gene disorder will likely harbor distinct combinations of mutations. Non-limiting examples of multi-gene disorders include autism, schizophrenia, intellectual disability, epilepsy, major depression, bipolar disorder, hyperlipidemia, autoimmune disease, multiple sclerosis, arthritis, lupus, inflammatory bowel disease, refractive error, cleft palate, hypertension, asthma, heart disease, type 2 diabetes, cancer, Alzheimer's disease and obesity.

The term “mutation” refers to a change in a nucleotide sequence (e.g., an insertion, deletion, inversion, duplication, or substitution of one or more nucleotides) of a gene. The term also encompasses the corresponding change in the complement of the nucleotide sequence, unless otherwise indicated.

### 2. PSC-based Models of Neurodegenerative Diseases

The present disclosure relates to methods for modulating cellular aging and/or progression of neurodegenerative diseases (e.g., AD). In certain embodiments, the methods induce cellular aging. In certain embodiments, the methods promote progression of neurodegenerative diseases (e.g., AD). The present disclosure also relates to methods and systems for modeling aging related neurodegenerative diseases (e.g., AD) in vitro. In certain embodiments, the neurodegenerative disease is AD, Parkinson's disease, Amyotrophic Lateral Sclerosis (ALS), or Huntington's disease.

In certain embodiments, the neurons can be obtained from in vitro differentiation of stem cells (e.g., human stem cells). In certain embodiments, the stem cell is a human stem cell. Non-limiting examples of human stem cells include human embryonic stem cells (hESC), human pluripotent stem cell (hPSC), human induced pluripotent stem cells (hiPSC), human parthenogenetic stem cells, primordial germ cell-like pluripotent stem cells, epiblast stem cells, F-class pluripotent stem cells, somatic stem cells, cancer stem cells, or any other cell capable of lineage specific differentiation. In certain embodiments, the human stem cell is a human pluripotent stem cell. In certain embodiments, the human stem cell is a human embryonic stem cell (hESC). In certain embodiments, the human stem cell is a human induced pluripotent stem cell (hiPSC). In certain embodiments, the stem cells are non-human stem cells, including, but not limited to, mammalian stem cells, primate stem cells, or stem cells from a rodent, a mouse, a rat, a dog, a cat, a horse, a pig, a cow, a sheep, etc. In certain embodiments, the neurons are cortical neurons.

In certain embodiments, the neurons exhibit genetic mutation of at least one gene that is associated with neurodegenerative disease. Non-limiting examples of such mutations include those utilized in models of AD, e.g., APP Swedish mutation K595N/M596L and PSEN M146V. Additional non-limiting examples include those utilized in models of Parkinson's disease, e.g., LRRK2 G2019S. In certain embodiments, the mutation of at least one gene that is associated with neurodegenerative disease results in increased production of amyloid-beta peptide. In certain embodiments, the mutation of at least one gene that is associated with neurodegenerative disease results in a change in the ratio of Aβ40 to Aβ42 peptide.

### 3. Inhibiting Protein Neddylation Pathways

In certain embodiments, the methods disclosed herein comprise inhibiting protein neddylation pathway. In certain embodiments, inhibiting protein neddylation pathway comprises knocking out or knocking down genes (e.g., UBA3, NAE1) that regulate protein neddylation pathway.

In certain embodiments, modulating protein neddylation pathway comprises administering a neddylation inhibitor to cells. Non-limiting examples of compounds that modulate neddylation include MLN4924, TAS4464, CSN5i-3, ZM223, NACM-OPT, Keap1-Nrf2-IN-4, WS-383, VII-31 and derivatives thereof.

MLN4924 refers to IUPAC name [(1S,2S,4R)-4-[4-[[(1S)-2,3-Dihydro-1H-inden-1-yl]amino]-7H-pyrrolo[2,3-d]pyrimidin-7-yl]-2-hydroxycyclopentyl]methyl sulfamic acid ester. MLN4924 is an inhibitor of NEDD8 activating enzyme (NAE).

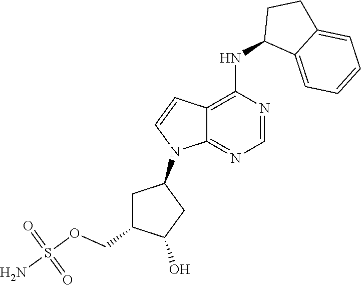 [SMILES: NS(=O)(=O)OC[C@@H]1C[C@@H](n2ccc3c(N[C@H]4CCc5ccccc54)ncnc32)C[C@@H]1O]

In certain embodiments, the neurons are contacted with between about 100 nM and about 10 μM, between about 100 nM and about 1 μM, or between about 100 nM and about 1 μM. In certain embodiments, the neurons are contacted with about 100 nm, about 1 μM, or about 10 μM neddylation inhibitor.

In certain embodiments, the neurons are contacted with the neddylation inhibitor for up to 10 days, up to 20 days, or up to 30 days, or up to 4 weeks, or up to 5 weeks, or up to 6 weeks. In certain embodiments, the neurons are contacted with the neddylation inhibitor for about 3 hours, about 1 day, about 2 days, about 3 days, about 5 days, about 7 days, about 10 days, about 20 days, or about 30 days, or about 4 weeks, or about 5 weeks, or about 6 weeks.

### 4. Identification of Genes Associated with Cellular Aging and/or Neurodegenerative Disease

The present disclosure relates to methods of identifying genes associated with cellular aging and/or progression of neurodegenerative disease. In certain embodiments, a target gene is selected based on differential expression in a model of neurodegenerative disease relative to healthy tissue. In certain embodiments, mutation is introduced at a target gene in a PSC model of neurodegenerative disease. In certain embodiments, functional activity is measured between the PSC model comprising further mutation at the target gene and compared to healthy tissue. In certain embodiments, the functional activity is cellular senescence, protein aggregation, DNA damage, decreased heterochromatin, and/or cell viability.

Any methods known in the art can be used to generate gene modifications in the PSC lines, e.g., hPSC lines. In certain embodiments, genome editing technique can be used to generate gene modifications in the PSC lines. For example, but not by way of limitation, a CRISPR/Cas9 system is employed to modify the genes. Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), and trans-activating crRNA (tracrRNA, binds to crRNA and forms an active complex with Cas9). The terms “guide RNA” and “gRNA” refer to any nucleic acid that promotes the specific association (or “targeting”) of an RNA-guided nuclease such as a Cas9 to a target sequence such as a genomic or episomal sequence in a cell. gRNAs can be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing). CRISPR/Cas9 strategies can employ a plasmid to transfect the mammalian cell. The gRNA can be designed for each application as this is the sequence that Cas9 uses to identify and directly bind to the target DNA in a cell. Multiple crRNA' s and the tracrRNA can be packaged together to form a single-guide RNA (sgRNA). The sgRNA can be joined together with the Cas9 gene and made into a plasmid in order to be transfected into cells. In certain embodiments, the CRISPR/Cas9 system comprising a Cas9 molecule, and a guide RNA (gRNA) comprising a targeting domain that is complementary with a target sequence of the targeted gene.

In certain embodiments, a zinc-finger nuclease (ZFN) system is employed for generating the gene modifications in the PSCs, e.g., hPSCs. The ZEN can act as restriction enzyme, which is generated by combining a zinc finger DNA-binding domain with a DNA-cleavage domain. A zinc finger domain can be engineered to target specific

DNA sequences which allows the zinc-finger nuclease to target desired sequences within genomes. The DNA-binding domains of individual ZFNs typically contain a plurality of individual zinc finger repeats and can each recognize a plurality of base pairs. The most common method to generate new zinc-finger domain is to combine smaller zinc-finger “modules” of known specificity. The most common cleavage domain in ZFNs is the non-specific cleavage domain from the type IIs restriction endonuclease Fokl. ZFN modulates the expression of proteins by producing double-strand breaks (DSBs) in the target DNA sequence, which will, in the absence of a homologous template, be repaired by non-homologous end-joining (NHEJ). Such repair may result in deletion or insertion of base-pairs, producing frame-shift and preventing the production of the harmful protein (Durai et ah, Nucleic Acids Res., 33 (18): 5978-90.) Multiple pairs of ZFNs can also be used to completely remove entire large segments of genomic sequence (Lee et ah, Genome Res., 20 (1): 81-9).

In certain embodiments, a transcription activator-like effector nuclease (TALEN) system is employed in generating the gene modifications in the PSCs, e.g., hPSCs. TALENs are restriction enzymes that can be engineered to cut specific sequences of DNA. TALEN systems operate on a similar principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome (Boch et ah, Nature Biotechnology;29(2): 135-6).

The genetic modification system disclosed herein can be delivered into the PSCs, e.g, hPSCs, using a retroviral vector, e.g, gamma-retroviral vectors, and lentiviral vectors. Combinations of retroviral vector and an appropriate packaging line are suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g, particles pseudotyped with VSVG, RD114 or GALV envelope and any other known in the art. Possible methods of transduction also include direct co-culture of the cells with producer cells, e.g, by the method of Bregni, et al. (1992) Blood 80:1418-1422, or culturing with viral supernatant alone or concentrated vector stocks with or without appropriate growth factors and polycations, e.g, by the method of Xu, et al. (1994) Exp. Hemat. 22:223-230; and Hughes, et al. (1992) J. Clin. Invest. 89:1817.

Other transducing viral vectors can also be used to generate gene modification in the PSCs, e.g., hPSCs, disclosed herein. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S. A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107: 77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for generating gene modifications in the PSCs, e.g., hPSCs. For example, a nucleic acid molecule can be introduced into the PSC by administering the nucleic acid in the presence of lipofection (Feigner et al., Proc. Natl. Acad. Sci. U.S. A. 84:7413, 1987; Ono et al., Neuroscience Letters 17:259, 1990; Brigham et al., Am. J. Med. Sci. 298:278, 1989; Staubinger et al., Methods in Enzymology 101: 512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of nucleic acid molecules into a cell.

### EXEMPLARY EMBODIMENTS

A. In certain non-limiting embodiments, the presently disclosed subject matter provides for a method of preparing an in vitro model of neurodegenerative disease comprising modulating protein neddylation in a population of neurons; wherein the population of neurons exhibit genetic mutation of at least one gene that is associated with neurodegenerative disease.

A1. The foregoing method of A, wherein modulating protein neddylation comprises exposing the population of neurons to a compound that modulates protein neddylation.
A2. The foregoing method of A-A1, wherein the at least one compound that modulates protein neddylation is selected from the group consisting of MLN4924, TAS4464, CSN51-3, ZM223, NACM-OPT, Keap1-Nrf2-IN-4, WS-383, VII-31, derivatives thereof, and combinations thereof.
A3. The foregoing method of A, wherein modulating protein neddylation comprises modifying expression of at least one gene which regulates protein neddylation pathways.
A4. The foregoing method of A3, wherein the at least one gene which regulates protein neddylation pathways is selected from the group consisting of UBA3, NAE1, and combinations thereof.
A5. The foregoing method of A-A4, wherein the mutation of at least one gene that is associated with neurodegenerative disease results in increased production of amyloid-beta peptide.
A6. The foregoing method of A-A4, wherein the mutation of at least one gene that is associated with neurodegenerative disease results in a change in the ratio of Aβ40 to Aβ42 peptide.
A7. The foregoing method of A-A4, wherein the at least one gene that is associated with neurodegenerative disease comprises the Amyloid-beta precursor protein (APP) gene. A8. The foregoing method of A6, wherein the mutation of the APP gene comprises K595N/M596L.
A9. The foregoing method of A-A4, wherein the at least one gene that is associated with neurodegenerative disease comprises the presenilin-1 (PSEN) gene.
A10. The foregoing method of A8, wherein the mutation of the PSEN gene comprises M146V.
A11. The foregoing method of A-A4, wherein the mutation of at least one gene that is associated with neurodegenerative disease results in increased production of N-ethylmaleimide sensitive factor (NSF) aggregates.
A12. The foregoing method of A-A4 or A11, wherein the at least one gene that is associated with neurodegenerative disease comprises the (LRRK2) gene.
A13. The foregoing method of A12, wherein the mutation of the LRRK2 gene comprises G2019S.
A14. The foregoing method of A-A13, wherein the neurodegenerative disease is Alzheimer's disease, Parkinson's disease, Amyotrophic Lateral Sclerosis (ALS), or Huntington's disease.
A15. The foregoing method of A-A14, wherein the neurons are obtained from in vitro differentiation of stem cells.
A16. The foregoing method of A15, wherein the stem cells are human stem cells.
A17. The foregoing method of A16, wherein the human stem cells are selected from the group consisting of human embryonic stem cells, human induced pluripotent stem cells, human parthenogenetic stem cells, human primordial germ cell-like pluripotent stem cells, human epiblast stem cells, human F-class pluripotent stem cells, and combinations thereof.
A18. The foregoing method of A-A17, wherein the neurons are cortical neurons.
B. In certain non-limiting embodiments, the presently disclosed subject matter provides for a method of identifying genes associated with cellular aging and/or progression of neurodegenerative disease comprising:
   a) obtaining a first population of neurons;
   b) obtaining a second population of neurons, and modifying expression of a test gene in the second population of neurons;
   c) measuring functional activity of the second population of neurons relative to the first population of neurons;
      wherein the first population of neurons and the second population of neurons exhibit genetic mutation of at least one gene that is associated with neurodegenerative disease; wherein a difference in the functional activity between the first population of neurons and the second population of neurons indicates that the test gene is associated with cellular aging and/or progression of neurodegenerative disease.
B1. The foregoing method of B, wherein the functional activity is selected from the group consisting of cellular senescence, protein aggregation, DNA damage, decreased heterochromatin, cell viability, and combinations thereof.
B2. The foregoing method of B-B1, wherein the neurodegenerative disease is Alzheimer's disease, Parkinson's disease, Amyotrophic Lateral Sclerosis (ALS), or Huntington's disease.
B3. The foregoing method of B-B2, wherein the mutation of at least one gene that is associated with neurodegenerative disease results in increased production of amyloid-beta peptide.
B4. The foregoing method of B-B2, wherein the mutation of at least one gene that is associated with neurodegenerative disease results in a change in the ratio of Aβ40 to Aβ42 peptide.
B5. The foregoing method of B-B2, wherein the at least one gene that is associated with neurodegenerative disease comprises the Amyloid-beta precursor protein (APP) gene.
B6. The foregoing method of B5, wherein the mutation of the APP gene comprises K595N/M596L.
B7. The foregoing method of B-B2, wherein the at least one gene that is associated with neurodegenerative disease comprises the presenilin-1 (PSEN) gene.
B8. The foregoing method of B7, wherein the mutation of the PSEN gene comprises M146V.
B9. The foregoing method of B-B2, wherein the mutation of at least one gene that is associated with neurodegenerative disease results in increased production of N-ethylmaleimide sensitive factor (NSF) aggregates.
B10. The foregoing method of B-B2 or B9, wherein the at least one gene that is associated with neurodegenerative disease comprises the (LRRK2) gene.
B11. The foregoing method of B10, wherein the mutation of the LRRK2 gene comprises G2019S.
B12. The foregoing method of B-B11, wherein the neurons are obtained from in vitro differentiation of stem cells.
B13. The foregoing method of B12, wherein the stem cells are human stem cells.
B14. The foregoing method of B13, wherein the human stem cells are selected from the group consisting of human embryonic stem cells, human induced pluripotent stem cells, human parthenogenetic stem cells, human primordial germ cell-like pluripotent stem cells, human epiblast stem cells, human F-class pluripotent stem cells, and combinations thereof.
B15. The foregoing method of B-B14, wherein the neurons are cortical neurons.
B16. The foregoing method of B-B15, wherein modifying expression of the test gene modulates protein neddylation in the second population of neurons.
C. In certain non-limiting embodiments, the presently disclosed subject matter provides for a composition for identifying genes associated with cellular aging and/or progression of neurodegenerative disease comprising a population of neurons, wherein the population of neurons exhibit genetic mutation at a test gene, wherein the population of neurons exhibit genetic mutation of at least one gene that is associated with neurodegenerative disease.
C1. The foregoing composition of C, wherein the neurodegenerative disease is Alzheimer's disease, Parkinson's disease, Amyotrophic Lateral Sclerosis (ALS), or Huntington's disease.
C2. The foregoing composition of C-C1, wherein the genetic mutation at the test gene modulates protein neddylation.
C3. The foregoing composition of C-C2, wherein the mutation of at least one gene that is associated with neurodegenerative disease results in increased production of amyloid-beta peptide.
C4. The foregoing composition of C-C2, wherein the mutation of at least one gene that is associated with neurodegenerative disease results in a change in the ratio of Aβ40 to Aβ42 peptide.
C5. The foregoing composition of C-C2, wherein the at least one gene that is associated with neurodegenerative disease comprises the Amyloid-beta precursor protein (APP) gene.
C6. The foregoing composition of C5, wherein the mutation of the APP gene comprises K595N/M596L.
C7. The foregoing composition of C-C2, wherein the at least one gene that is associated with neurodegenerative disease comprises the presenilin-1 (PSEN) gene.
C8. The foregoing composition of C7, wherein the mutation of the PSEN gene comprises M146V.
C9. The foregoing composition of C-C2, wherein the mutation of at least one gene that is associated with neurodegenerative disease results in increased production of N-ethylmaleimide sensitive factor (NSF) aggregates.
C10. The foregoing composition of C-C2 and C9, wherein the at least one gene that is associated with neurodegenerative disease comprises the (LRRK2) gene.
C11. The foregoing composition of C10, wherein the mutation of the LRRK2 gene comprises G2019S.
C12. The foregoing composition of C-C11, wherein the neurons are obtained from in vitro differentiation of stem cells.
C13. The foregoing composition of C12, wherein the stem cells are human stem cells.
C14. The foregoing composition of C13, wherein the human stem cells are selected from the group consisting of human embryonic stem cells, human induced pluripotent stem cells, human parthenogenetic stem cells, human primordial germ cell-like pluripotent stem cells, human epiblast stem cells, human F-class pluripotent stem cells, and combinations thereof.
C15. The foregoing composition of C-C14, wherein the neurons are cortical neurons.

### EXAMPLES

The present disclosure will be better understood by reference to the following Example, which is provided as exemplary of the presently disclosed subject matter, and not by way of limitation.

### Example 1—Genome-Wide CRISPR Screen Identifies Neddylation as a Regulator of Neuronal Aging and AD-neurodegeneration.

Aging is the biggest risk factor for the development of AD, but it not known whether the aging process directly contributes to AD initiation or progression. Understanding the how cellular age is regulated is also important because pluripotent stem cell (PSC) derived neurons are transcriptionally and functionally young and this may limit their utility in modelling the late onset degenerative phase of AD. It was previously shown that age can be directly programmed into PSC derived neurons through the ectopic expression of Progerin raising the question of what genes and pathways regulate this process during normal aging. To address this, past studies either performed RNAseq on postmortem brain tissue from young versus old individuals or generated induced neurons via direct reprogramming from primary fibroblasts donated by individuals of varying ages. Although neurons generated via direct reprogramming may retain a fibroblast-related aging signature, it has been challenging to identify which changes have a deterministic impact on cellular age.

### Methods

hESC culture and differentiation. Engineered human embryonic stem cells (H9; WA-09) were maintained on Vitronectin coated plates in E8 medium and passaged twice a week using EDTA. For differentiation to cortical neurons, the PSCs were dissociated to single cells using Accutase and replated onto Matrigel coated dishes at a density of 300,000 k/cm² in E8 medium supplemented with ROCK inhibitor (Y-27632; 10 μM). The following day (DIV=0) culture medium was replaced with E6 containing SB431542 (10 μM), LDN193189 (100 nM) and XAV939 (2 μM). Differentiation media was changed daily and XAV939 removed after 3 days. At 10 DIV the media was changed to neurobasal supplemented with N2 and B27 and the monolayer was maintained for an additional 10 days. On day 20 after neural induction cells were dissociated using Accutase and replated onto poly-L-ornithine/fibronectin/laminin-coated plates. Neurons were maintained in Neurobasal medium supplemented with BDNF, ascorbic acid, GDNF, CAMP, L-glutamine and B-27 supplement. DAPT was also added to the culture media until 30 days DIV.

Cell line engineering. WA-09 were sequentially engineered to generate the cell lines for this study. First an inducible Cas9 construct was knocked into the AAVS1 locus as described in Gonzalez et al. but with a hygro-resistant Cas9 donor plasmid instead of the puromycin resistant donor. After checking for the correct insertion of the iCas9 construct the newly established iCas9 cell line was engineered to insert the APP^{swe/swe} mutation as described in Paquet et al. The maintenance of an intact karyotype was confirmed after each engineering step.

Whole genome CRISPR Cas9 screen in PSC-derived neurons. The Brunello human CRISPR Knockout Pooled Library was used for this screen; this library includes 4 guide RNAs for 19,114 genes as well as 1000 non targeting controls. Stem cell culture, transduction, selection, and differentiation of the isogenic stem cell pair was done in parallel. To perform the screen PSCs were dissociated with Accutase and a total of 250 million cells per line were replated at a density of 150,000/cm² in E8 medium with ROCK inhibitor (Y-27632; 10 μM). The whole genome lenti-guide RNA library was added during the replating step at an MOI of 0.3-0.5. The virus was removed 16-18 h post transduction and fresh E8 medium added to the culture plate. The following day transduced cells were selected by adding 0.4 μg/ml puromycin to the E8 medium. After selection, PSCs were dissociated with Accutase, the culture plates were pooled and a total of 116 million cells used for differentiation to ensure that 1000× guide representation was maintained throughout. PSCs were differentiated as described in the ‘hESC culture and differentiation’ subsection. After differentiation (DIV 20) cultures were dissociated using Accutase to generate a single cell suspension for each cell line and the cells were split to give triplicate samples for the screen with a total of 91 million cells per replicate. The T=0 control samples were harvested immediately whereas the endpoint samples were replated at a density of 200,000/cm² in neurobasal supplemented with N2, B27 and ROCK inhibitor (Y-27632; 10 μM). Doxycycline (2 μg/ml) was added to half the culture plates to induce Cas9 expression. Cells were treated with doxycycline for a total of 48 h before switching to neural maintenance media with DAPT until DIV 30. From DIV 30 onwards half the culture media was replaced every 2-3 days. Neurons were harvested at DIV 65. To remove any dead cells from the culture the monolayer was washed 2× with PBS followed by a 5 min incubation in EDTA at RT. Then, the neuronal monolayer was scrapped off the culture dish, pelleted and snap frozen. Cell Pellets from pooled screen were lysed, and genomic DNA was extracted (Qiagen) and PCR amplified to add Illumina adapters and multiplexing barcodes. Amplicons were quantified by Qubit and Bioanalyzer (Agilent) and sequenced on Illumina HiSeq 2500.

Data analysis for Pooled CRISPR screen. Sequencing reads were aligned to the screened library and the CRISPR screen was analyzed using the MAGECK-MLE pipeline as previously described. To calculate the beta scores for each gene the representation of guide RNAs in the endpoint samples (DIV65+Dox) was compared to either the DIV20 or DIV65−Dox samples. Non targeting sgRNAs were used for normalization. Hit genes were identified by comparing the beta scores in the wild-type viability screen with the beta scores in the App^{swe/swe} viability screen. Genes with a beta score<0, FDR<0.3 and Pval<0.05 were considered viability genes. Essential genes met these criteria in both genotypes. Candidate age regulators met these criteria in the APP^{swe/swe} genotype. The hit list was filtered to exclude genes that had a viability phenotype in the wild-type neurons (Beta score^{WT} was greater than 1.5 standard deviations from the mean or <0 with an FDR^{WT}<0.3 and Pval^{WT}<0.05).

RNA extraction and qPCR. RNA was extracted using the Zymo RNA Micro Kit and total of lug of RNA was used to generate cDNA using iScript (BioRad). Realtime PCR was performed using SSoFAST EvaGreen Mix (BioRad) in a BioRad CFX96

Thermal Cycler. The manufacturers protocol was used for all steps.

TABLE 1
Primers used in this study.

| Target | Sequence | Sequence ID |
|---|---|---|
| Cas9 Fwd | CCGAAGAGGTCGTGAAGAAG | SEQ ID NO: 1 |
| Cas9 Rev | GCCTTATCCAGTTCGCTCAG | SEQ ID NO: 2 |
| FOXG1 Fwd | CAACGGCATCTACGAGTTCA | SEQ ID NO: 3 |
| FOXG1 Rev | TGTTGAGGGACAGATTGTGG | SEQ ID NO: 4 |
| GAPDH Fwd | ATGTTCGTCATGGGTGTGAA | SEQ ID NO: 5 |
| GAPDH Rev | AGGGGTGCTAAGCAGTTGGT | SEQ ID NO: 6 |
| OCT4 Fwd | CTGGGTTGATCCTCGGACCT | SEQ ID NO: 7 |
| OCT4 Rev | CCATCGGAGTTGCTCTCCA | SEQ ID NO: 8 |
| PAX6 Fwd | GTCCATCTTTGCTTGGGAAA | SEQ ID NO: 9 |
| PAX6 Rev | TAGCCAGGTTGCGAAGAACT | SEQ ID NO: 10 |
| TUBB3 Fwd | CGATGCCATGCTCATCAC | SEQ ID NO: 11 |
| TUBB3 Rev | CCCAGTATGAGGGAGATCGT | SEQ ID NO: 12 |

Immunocytochemistry. Cells were fixed in 4% paraformaldehyde for 10 mins then permeabilized in PBS+0.3% Triton. Cells were blocked in 5% donkey or goat serum for 1 h. Primary antibody incubation was performed overnight. Primary antibodies used in this study were: p-ATM (S1981) (Thermo Fisher; MA1-2020), Cas9 (Cell Signaling Technologies; 14697S), COL1A1 (R&D; AF6220), FOXG1 (Takara; M227), Ki67 (Dako; M7240), MAP2 (Thermo Fisher; PA1-16751), NANOG (Cell Signaling Technologies; 4903S), PAX6 (Biolegend; 901301), TAU (Thermo Fisher; MN1000), p-TAU (S235) (Thermo Fisher; PA5-104785). For all image quantifications images were taken from 3 individual wells and averaged. This was repeated three times with neurons from independent differentiations. Volocity was used to count MAP2+ and Ki67+ cells with pyknotic. DAPI^{bright} nuclei excluded from the cell count. FIJI was used for TAU and pATM quantifications.

Aβ ELISA. To quantify amyloid peptide production cell culture media was harvested after 48 h and the culture media was briefly centrifuged to remove any cellular debris. Quantification was performed using the Mesoscale Discovery Assay kit (K15200E-2) according to the manufacturer's instructions. A total of 25 μl of culture medium was assayed. For each condition, the reported values represent the average of 3-4 culture wells per differentiation/experiment. For normalization, protein was extracted and quantified from neurons of matched age/gene knockout. The mean change in total protein between −Dox and +Dox samples from 3 independent differentiations/experiments was used to normalize total Aβ measurements.

Western blotting. Samples for western blotting were harvested, pelleted and snap frozen. Cell pellets were resuspended in RIPA buffer supplemented with Halt protease and phosphatase inhibitors (ThermoFisher) followed by centrifugation to clarify the sample. Protein concentration was quantified using the Precision Red Advanced Protein Assay according to manufacturer's instructions and equal amounts of protein were mixed with NuPAGE LDS Sample Buffer and NuPAGE Sample Reducing Agent and heated to 72 degrees for 10 mins. A total of 5-20 μg of protein was separated on NuPAGE Novex 4-12% Bis Tris gels and transferred by wet blotting onto PVDF membranes. Membranes were blocked in 5% milk protein or 5% BSA when probing for the phospho-Tau. Primary antibodies used for this study were: β-ACTIN (Sigma, A2228-100UL), BAG1 (Santa Cruz, sc-33704), BAG3 (Abcam, ab47124), Cas9 (Cell Signaling Technologies; 14697S), GAPDH-HRP (Santa Cruz Biotech, sc-47724 HRP), LMNB1 (Abcam, ab16048), NAE1(Cell Signaling Technologies, 14321S), NEDD8 (Abcam, ab81264), p21 (Cell Signaling Technologies, 2947), TAU (Dako, A0024), p-TAU(S202/T205) (Thermo Fisher; MN1020), UBA3 (Abcam, ab124728). Band intensity was visualized using BioRad ChemiDoc XRS+molecular imager. After imaging the membrane was re-probed with either GAPDH or β-ACTIN antibodies for normalization. Band intensity was quantified using Fiji.

Viability assays. Viability assays were performed in 96 well plates using the PrestoBlue Cell Viability Reagent or CCK8. Presto blue reagent was diluted 1:10 in neural maintenance media and 85 μl was applied to each well. For the CCK8 assay the assay reagent was prepared as described by the manufacturer with 110 μl used per well. Culture plates were incubated with the assay reagent for 2 h at 37 degrees before assaying. For the secondary validation experiments, each well was normalized to the mean absorbance of the no doxycycline control wells and the technical replicates averaged to give a single value for each differentiation/experiment.

Secondary validation. Secondary validation was performed in array in 96 well plates. Cells for secondary validation were differentiated as described in the ‘hESC culture and differentiation’ subsection. At DIV 20 neurons were dissociated and plated at 150,000/cm² in 96 wells. Three replicates were plated for each experiment/independent differentiation. For each guide RNA there were 4 conditions: WT neurons+guideRNA lentivirus, WT+guideRNA lentivirus+doxycycline, APP^{swe/swe} neurons+guideRNA lentivirus, APP^{swe/swe} neurons+guideRNA lentivirus+doxycycline. Unconcentrated virus was applied at a 1:30 dilution at DIV20 and DIV21. Doxycycline (2 ug/mL) was also added for the first 48 h. On DIV22 1 ug/mL puromycin was added to the cultures for 48 h to select for neurons transduced with the guideRNA of interest. Neurons were then maintained as previously described until DIV60 then assayed using the PrestoBlue Cell Viability Reagent as described in the ‘Viability assays’ subsection.

Generation of lentiGuide RNA viruses. GuideRNAs used for secondary validation were the top scoring guide RNA from the WGS. A list of guideRNA sequences used for this study can be found in Table 2. Guide RNAs were cloned into the lentiGuide-Puro plasmid (Addgene 52963) as described by the Zhang lab. For viral packaging, the lentiGuide-Puro plasmid and packaging plasmids (psPAX2; Addgene 12260 and pMD2.G; Addgene 12259) were transfected into 293T cells using X-tremeGENE HP (Sigma) in a 10:10:1 molar ratio, respectively. Virus particles were harvested after 48 h.

TABLE 2
sgRNA used in this study.

| Target | Sequence | SEQ ID |
|---|---|---|
| Non- | AGCGCAGATAGCGCGTATCA | SEQ ID NO: 13 |
| targeting | | |
| TOMM22 | ACAGCTAGATGAGACCCTGT | SEQ ID NO: 14 |
| SUFU | TGGCCCGCAGAGTTAATGCA | SEQ ID NO: 15 |
| PPIAL4E | CCAGGGTTTATGTGTCAGGG | SEQ ID NO: 16 |
| NUDT6 | GGATATGCTTCACATCAAGT | SEQ ID NO: 17 |
| NAE1 | TCAAAGAAGCAGTATCGGCA | SEQ ID NO: 18 |
| CEP170B | GGAACACACACCATACTGCG | SEQ ID NO: 19 |
| UBA3 | CATTCCAGGCAGAATCACCC | SEQ ID NO: 20 |
| DNAJB11 | ATCTTATAGAAATCTCGTCT | SEQ ID NO: 21 |
| VPS36 | TCAGTCGGTGTGTACTAAGA | SEQ ID NO: 22 |
| PPP1CB | GATCCAGATAAGGATGTGCA | SEQ ID NO: 23 |
| FAM76B | CCTTTCGAGGAGCTCTCCCA | SEQ ID NO: 24 |
| SLITRK5 | CGACATGCGCTCCATTAAGT | SEQ ID NO: 25 |
| ATXN2 | AATAGAGAAGTCATATCCTG | SEQ ID NO: 26 |
| ANKRD13A | GGAAAGGTCGAAGCGTTCGG | SEQ ID NO: 27 |
| NDUFAF7 | GACACCTTTCATATACACTG | SEQ ID NO: 28 |
| PAWR | AGTACGAAGATGATGAAGCA | SEQ ID NO: 29 |
| ARHGAP1 | CTTGCGGTCAAACTCCCGGT | SEQ ID NO: 30 |
| NOVA1 | GATGCGATCTGGATTAACGG | SEQ ID NO: 31 |
| GUK1 | GGCATGCTCGATGAAGTCGC | SEQ ID NO: 32 |
| SBF1 | TTGACCGGAGAAATGCGGAA | SEQ ID NO: 33 |
| PUM1 | TCTTAGACAGGAATCGCCCG | SEQ ID NO: 34 |
| NAA20 | ACCAAGGCGTCGAAATTCTG | SEQ ID NO: 35 |
| TRAPPC2L | GGTGCACACATCTCTGGACG | SEQ ID NO: 36 |
| TBC1D13 | GTGGCAAAGGTGTAGTAGAG | SEQ ID NO: 37 |
| URGCP | CCGAGTCAGGCAACACGAGT | SEQ ID NO: 38 |
| ATAD3B | CCGGCCGGTCTCCCAAACCG | SEQ ID NO: 39 |
| MASTL | GAAGGTGTGGGATTGACTAC | SEQ ID NO: 40 |
| COL6A6 | GTTTAGCGATACCTATCACC | SEQ ID NO: 41 |
| CCDC37 | CAGTGTTAACTCCACACCAG | SEQ ID NO: 42 |

Flow Cytometry. Neuronal cultures were dissociated to single cell suspensions using Accutase (Innovative Cell Technologies) supplemented with Neuron Isolation Enzyme for Pierce™ (Thermo 88285) solution at 1:50. Single cell suspensions were stained with Zombie UV™ Fixable Viability Kit (Biolegend 423107) at 1:2500 in PBS for 15 minutes at room temperature, followed by fixation in 4% Paraformaldehyde for 10 minutes (4° C.). Cells stained with CellEvent Senescence Green (Thermo C10840) were done so at 1:250 in assay buffer for 2 hours at 37° C. For intracellular probes, cells were permeabilized in 0.5% triton-x for 10 minutes (4° C.), and blocked in 5% BSA for 10 minutes (4° C.). Cells were stained with H3k9me3-PE antibody (Cell Signaling Technologies #13969S) diluted 1:200, and Proteostat (Enzo Life Sciences ENZ-51023-KP050) diluted 1:2500, in 5% BSA in PBS for 30 minutes at 4° C. Cells were analyzed on the Cytek Aurora Flow Cytometer. Experiments were repeated with cells from 3 independent differentiations.

Statistics and Reproducibility. Exact number of replicates, statistical test used, and error bars are defined in the relevant figure legends. Independent replicates consisted of an independent differentiation for neurons or independent passage for stem cells.

### Results

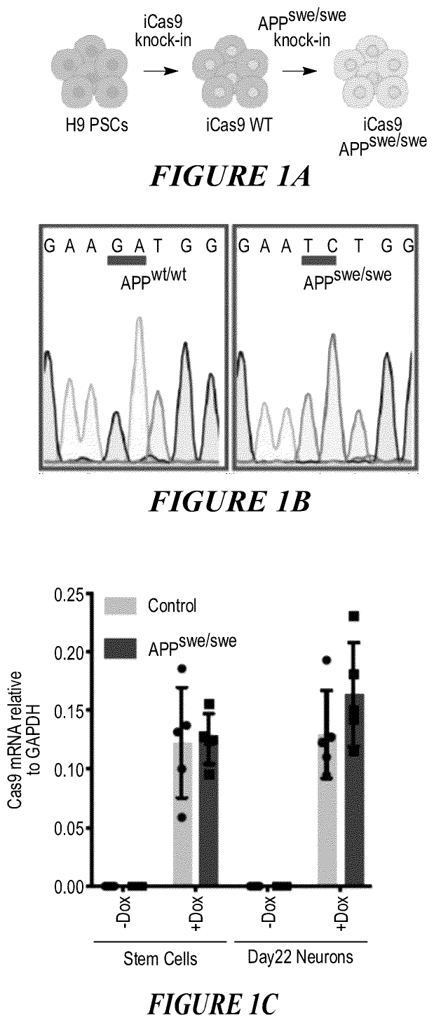

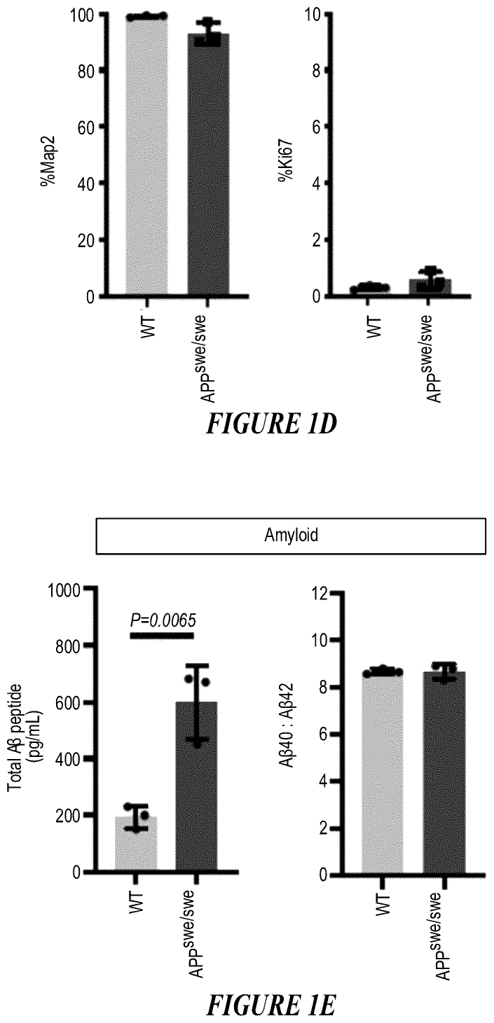

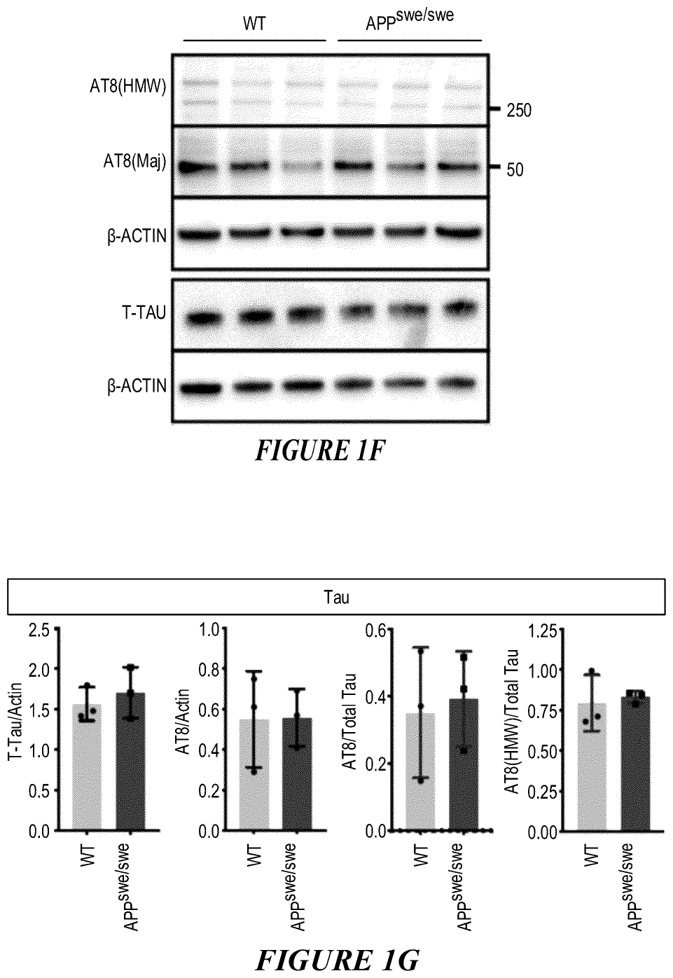

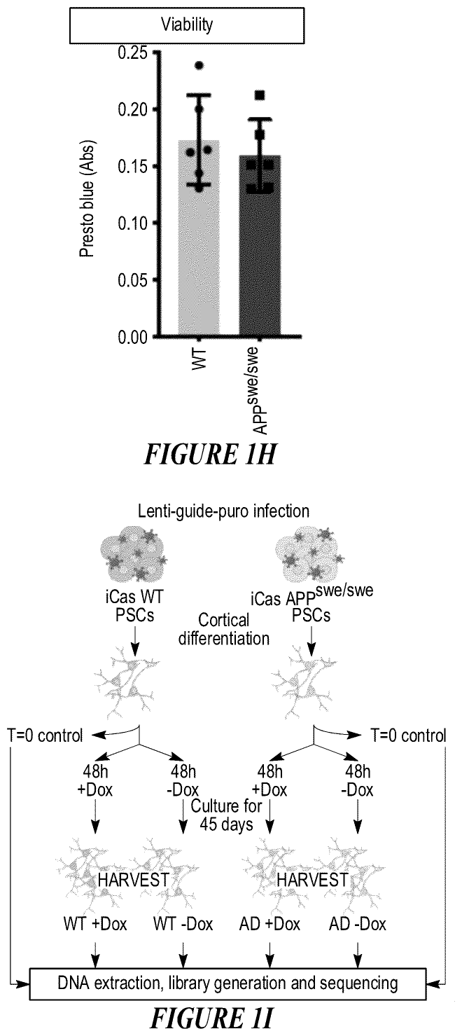

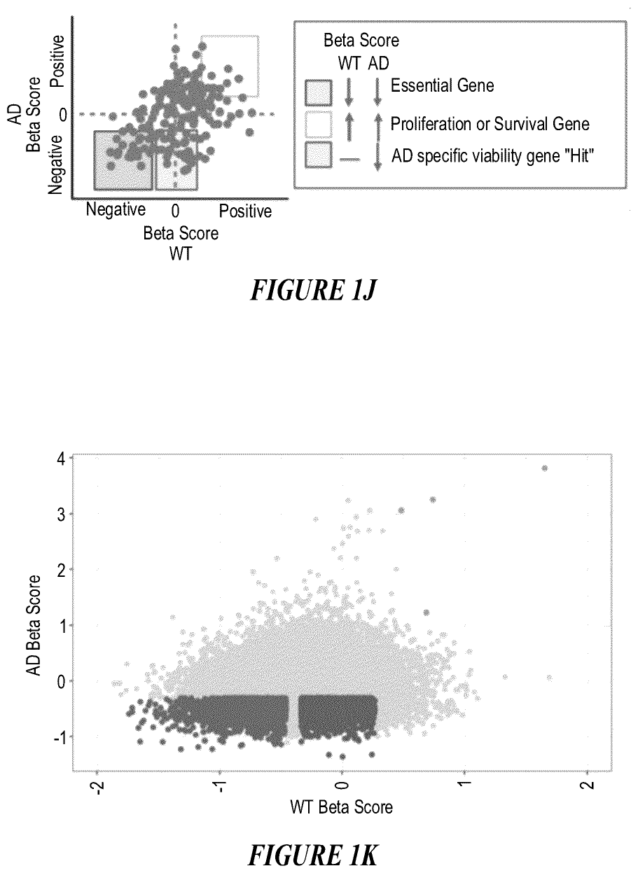

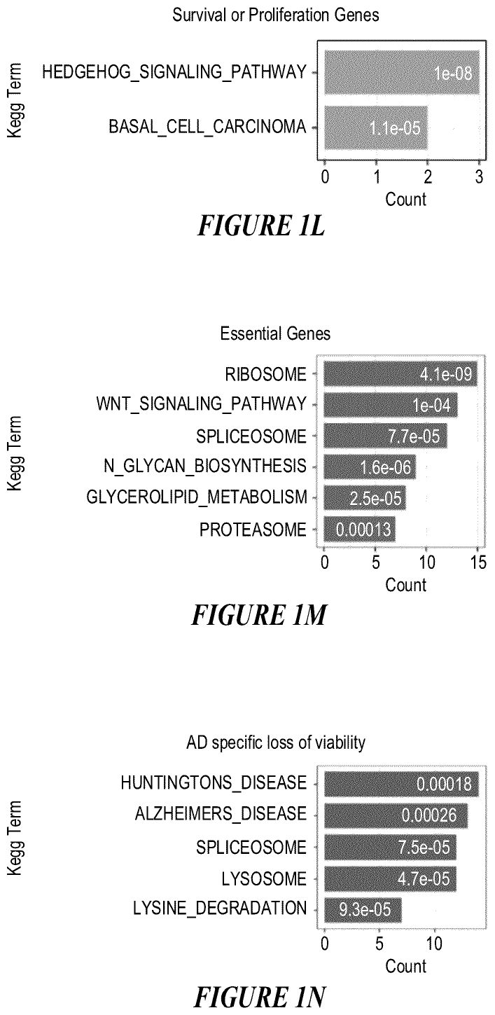

To define genes whose loss of function can drive the aging process a functional screening approach was developed that could be performed at whole genome scale with a single readout. Biomarker studies have shown that individuals with AD show a decades long age-associated progression of AD pathologies beginning with disordered amyloid precursor protein (APP) processing followed by Tau mislocalization and aggregation and finally, neuronal loss and cognitive dysfunction. Therefore, neuronal death was selected as an age and AD-dependent cellular readout of the whole genome screen (WGS). An isogenic stem cell model of familial AD (fAD) that was amenable to whole genome CRISPR/Cas9 screening was generated by sequential genome engineering of human PSCs (FIG. 1A). First, a dox inducible Cas9 (iCas9) was knocked into the AAVS1 safe-harbor locus as previously described. Then, the iCas9 line was further engineered to include a homozygous, two base pair mutation (GA>TC; APP Swedish mutation) in APP (FIG. 1B). In patients, this autosomal dominant mutation is sufficient to trigger early onset AD.

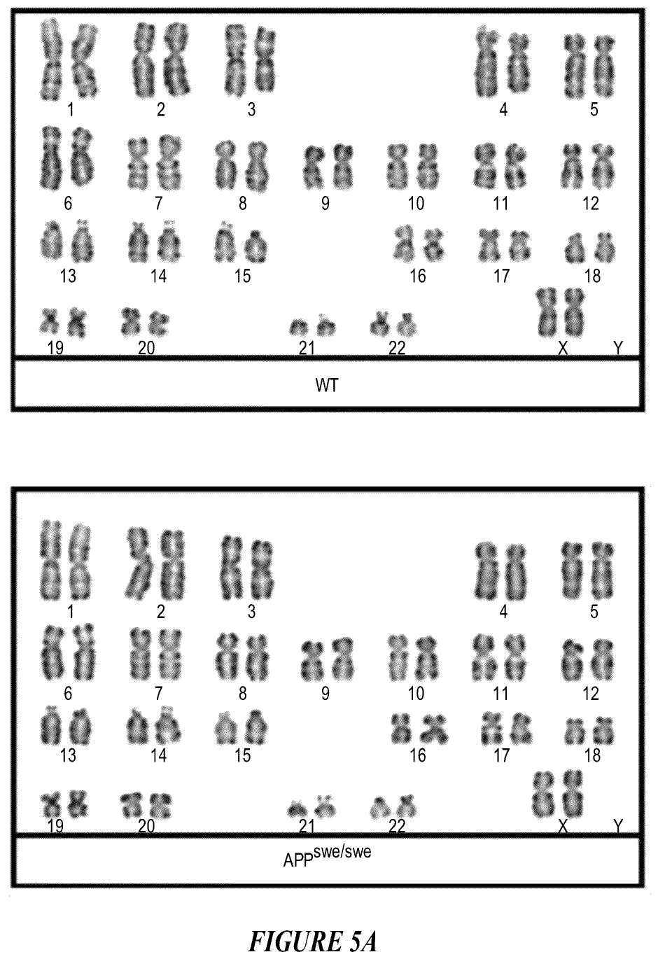

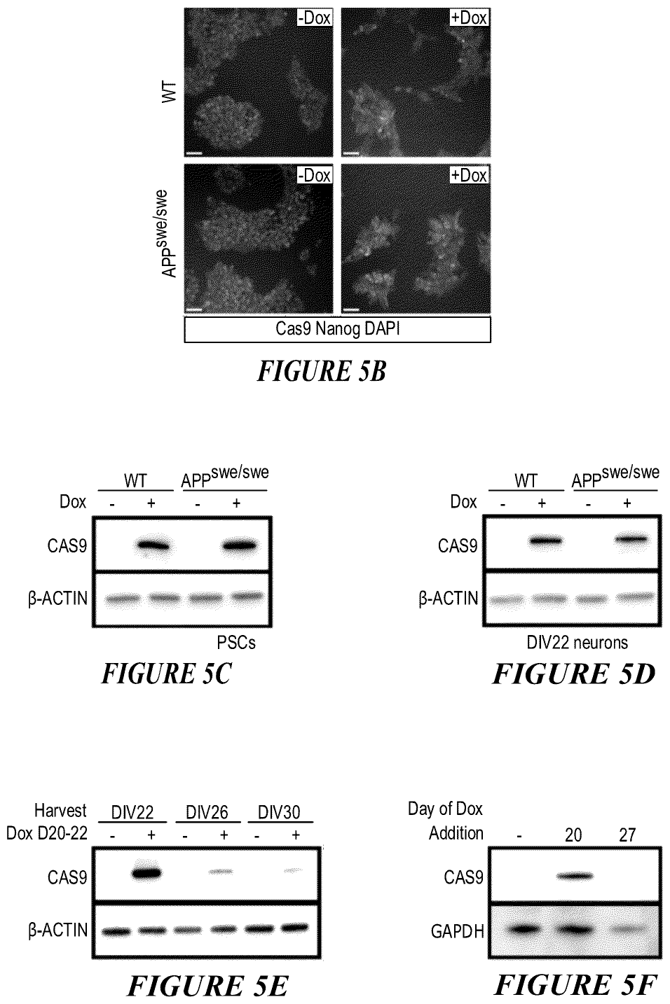

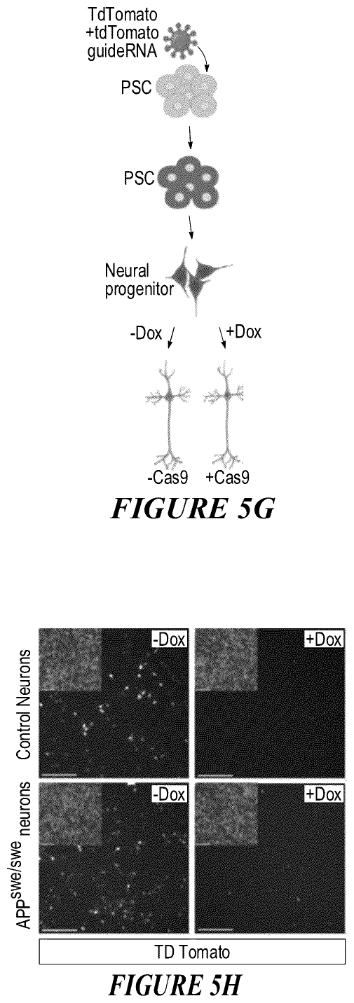

Both the Control and APP^{swe/swe} engineered cell lines had a normal karyotype and showed equivalent induction of Cas9 upon the addition of doxycycline (FIG. 1C, FIG. 5A-5D) with Cas9 levels declining rapidly after doxycycline removal (FIG. 5E). Importantly, Cas9 expression could be efficiently induced at equivalent levels in PSCs and at the endpoint of the cortical differentiation protocol (DIV20) but not in more mature neurons where the inducible transgene was silenced (FIG. 5F). Finally, as a proof of principle, the induction of Cas9 at DIV20 was shown to be sufficient to ablate TD-tomato expression in neurons that had been differentiated from TD-tomato+ stem cells transduced with a lentiviral guideRNA targeting the Td-Tomato gene. (FIG. 5G-5H). This experimental set up mirrored the conditions proposed for whole genome CRISPR screen.

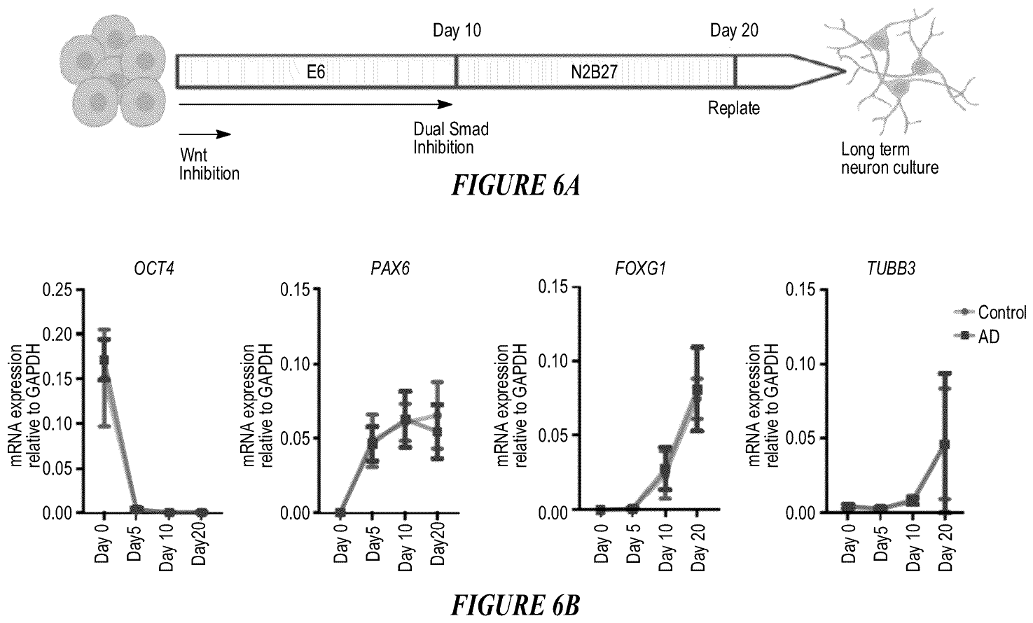

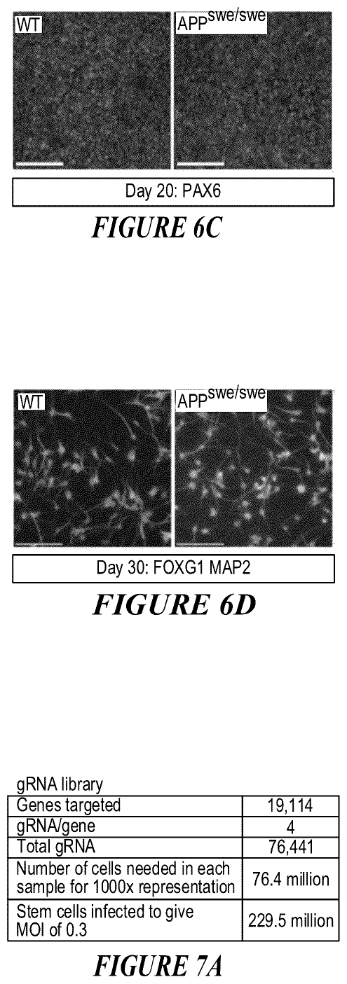

Further, it was confirmed that both lines could be differentiated into cortical neurons with equivalent efficiency (FIG. 6A-6D). Cortical progenitors were dissociated after 20 days and replated at low density in the presence of DAPT to induce terminal differentiation to neurons. By DIV30>99% of cells were MAP2+ in both cell lines with less than about 1% of cycling Ki67+ cells remaining (FIG. 1D). Having a near pure population of neurons without remaining neural progenitor cells (NPCs) allows for performing a depletion screen based on the differential survival of neurons of different genotypes rather than decreased proliferation of NPCs.

Next, experiments were performed to characterize amyloid, Tau and neuronal loss phenotypes in both genotypes at baseline. Knock-in of the APP^{swe/swe} mutation resulted in approximately 3-fold more total Aβ (Aβ38+Aβ40+Aβ42) than the isogenic control neurons without altering the ratio of Aβ40 to Aβ42 (FIG. 1E). This is consistent with the increase in total Aβ peptide production in iPSC derived APP^{swe/swe} cortical neurons reported by Kwart et al. However, no difference in levels of Tau or phospho-Tau between the WT and APP^{swe/swe} neurons (FIG. 1F,G) were discovered. This is consistent with the hypothesis that incorporating cellular age into AP-PSC models is important for the development of late onset phenotypes. Also, cell viability at DIV65 was also assayed, and it was confirmed that there was no difference in the viability of APP^{swe/swe} neurons relative to the isogenic control (FIG. 1H). In summary, the presently disclosed PSC model of AD faithfully recapitulates the initial biochemical changes in APP processing but not the late-stage neurodegenerative pathologies seen in AD patients.

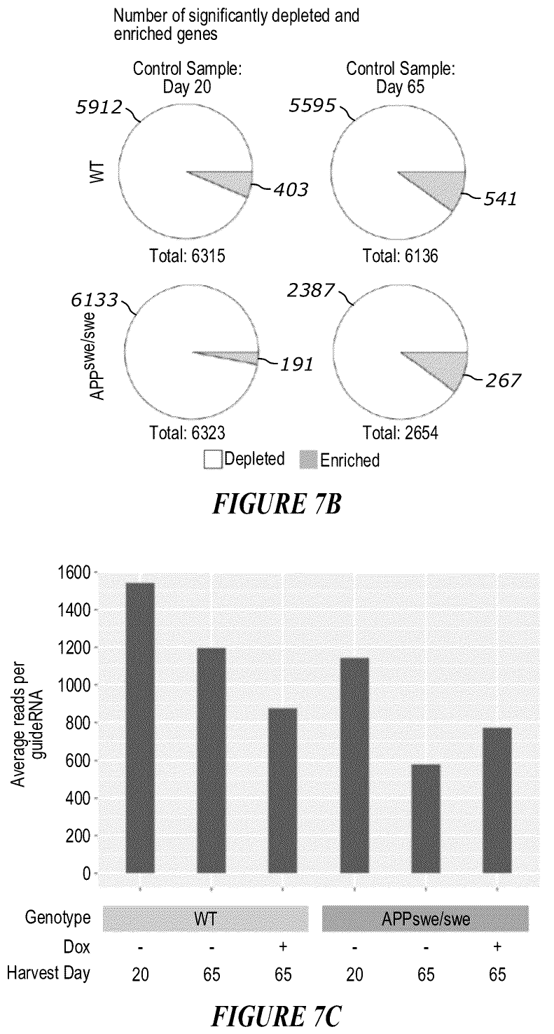

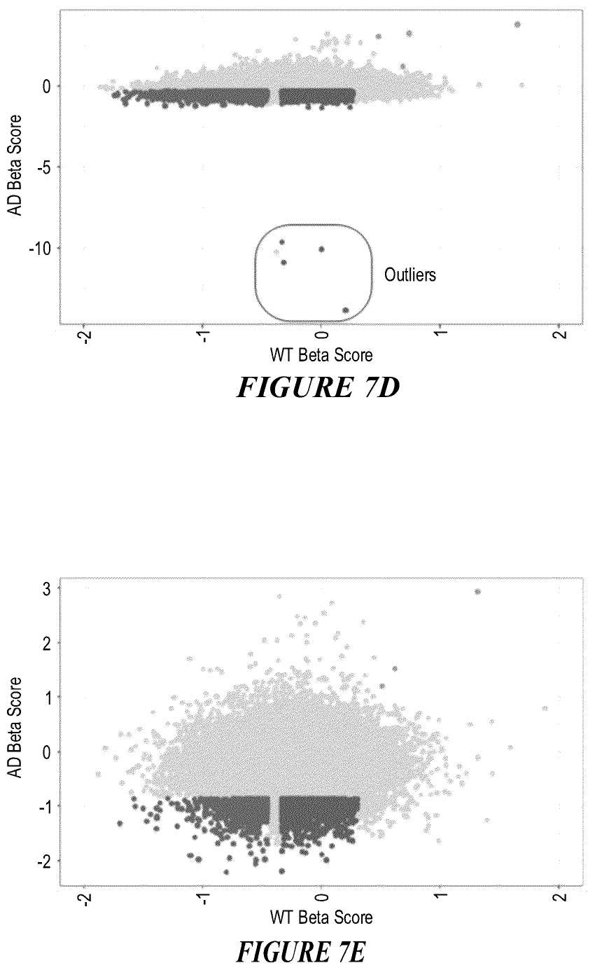

The workflow and experimental design of the paired whole genome CRISPR/Cas9 viability screen are summarized in FIG. 1I and described in detail in the methods section. The MAGeCK-MLE pipeline was used to determine gene essentialities and calculate the beta score for each gene. This metric has the advantage that it can be used to compare gene essentialities between different conditions, experiments, or cell lines. In view of minimal loss of viability at baseline across both genotypes based on the WGS performed in postmitotic neurons, proliferation or survival genes was not expected to be seen in this screen. Indeed, 90-97% of genes whose representation significantly changed over the course of the screen were depleted (negative beta score) and only 3-10% were significantly enriched (positive beta score) across all conditions (FIG. 7B). Also of note is the decrease in the mean guide RNA representation in the endpoint APP^{swe/swe}-Dox condition (FIG. 7C). Therefore, the analysis was focused on using the T=0 samples as the control sample for guideRNA representation.

A scatter plot was used to visualize the beta scores for each gene in the two different genotypes (FIG. 1J). This allows the separation of genes into several different categories: essential genes that that have a negative score in both cell lines (green), survival or proliferation genes that have a positive score in both cell lines (yellow) and hit genes that have a negative beta score only in the APP^{swe/swe} neurons (blue) (FIG. 1K; plot without outlier removal FIG. 7D). Five outliers were removed as none of these outliers was a hit when the guide representation was compared to the Day 65-Dox sample (FIG. 7E).

For each of the hit categories, the top 1000 genes were selected and a KEGG pathway analysis was run at an FDR<0.01. There were only 4 genes whose loss of function resulted in an increase in guide RNA representation in both genotypes. KEGG analysis shows that 3 of these genes fall within the hedgehog signaling pathway (FIG. 1L). The list of essential genes (depleted in both AD and isogenic control neurons) was significantly upregulated in genes associated with the ribosome, the spliceosome and proteasome (FIG. 1M). Genes associated with these pathways have been repeatedly found to be essential across a range of cell types and in screens performed using both siRNAs and the Cas9 platform. Other essential pathways identified in this screen include Wnt Signaling, N glycan biosynthesis and glycerolipid biosynthesis, which likely reflect more neuron-specific biology.

Pathway analysis of genes that were significantly depleted in the APP^{swe/swe} neurons and not in the WT neurons showed a significant overrepresentation of genes associated with both AD and Huntington's disease (FIG. 1N). This provides additional validation of the screening platform. Additional pathways identified include spliceosome, the lysosome and lysine degradation. Lysosome dysfunction has been identified in human AD brain and impairment in chaperone mediated autophagy arising from deletion of the lysosomal protein LAMP2A accelerated pathology in mouse models of AD in a manner reminiscent of aging. In addition, several AD risk genes identified in GWAS studies are involved in the endolysosomal system. Aberrant splicing has also been linked to AD. Although the link to lysine degradation is less clear, it is important to note that three of the genes in this category are involved in H3K9 methylation (SUV39H1, SUV39H2 and SETBD1). Loss of heterochromatin can induce accelerated aging phenotypes, is a hallmark of aging and can contribute to endogenous retrovirus activation.

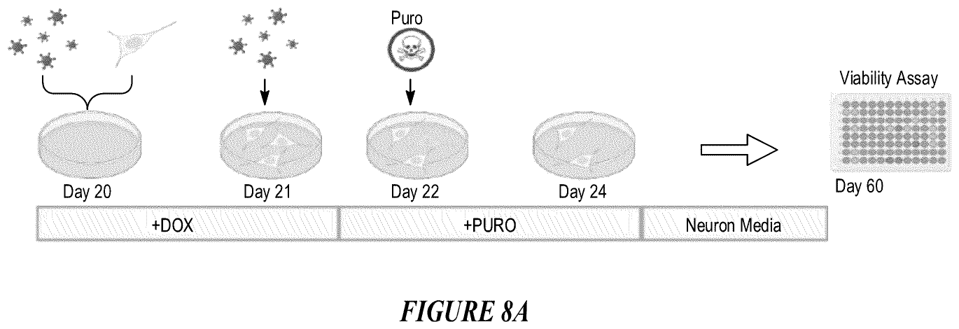

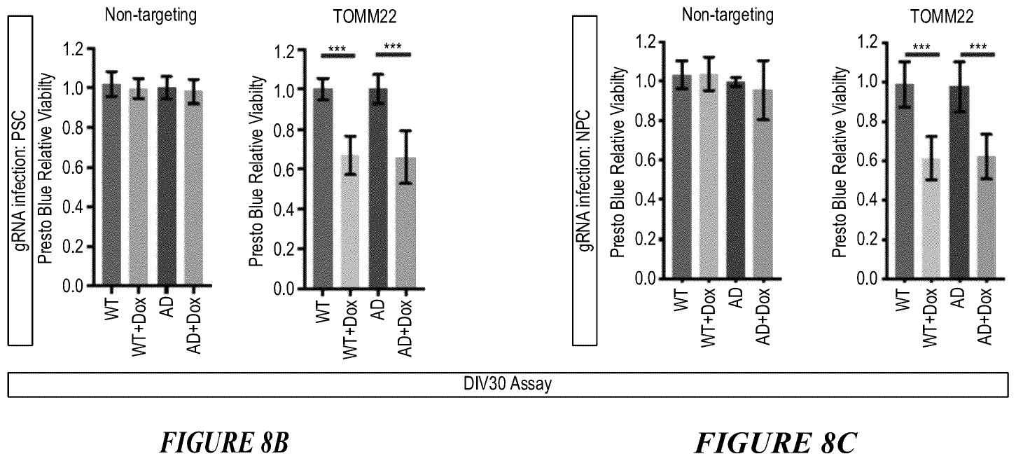

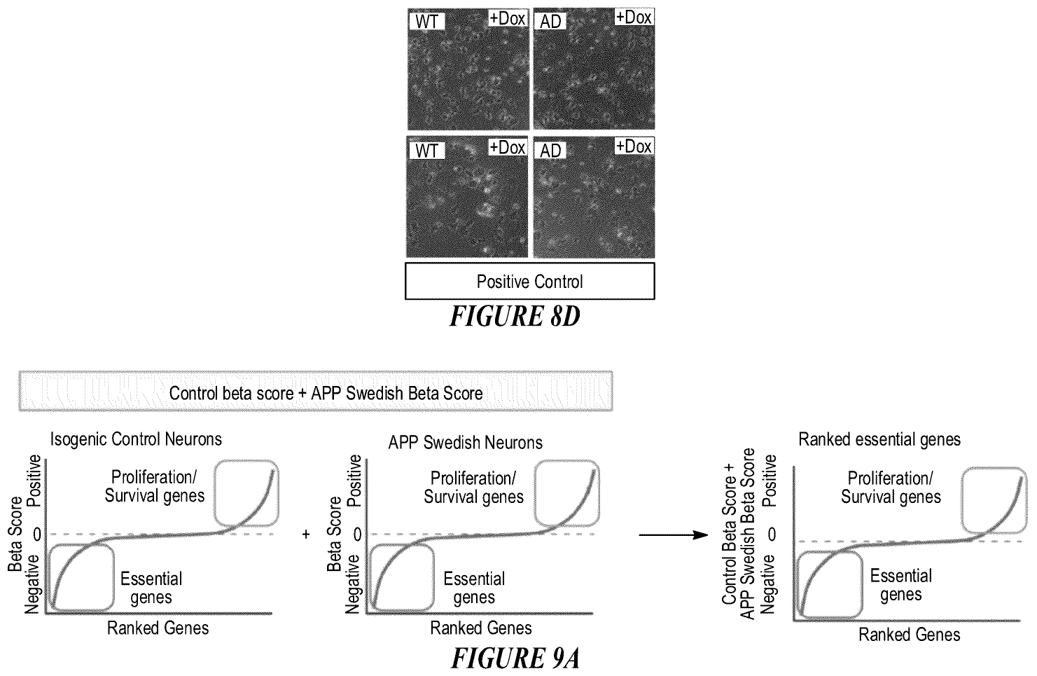

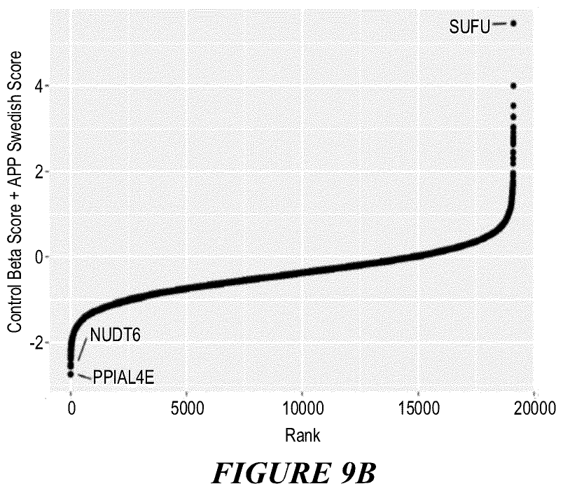

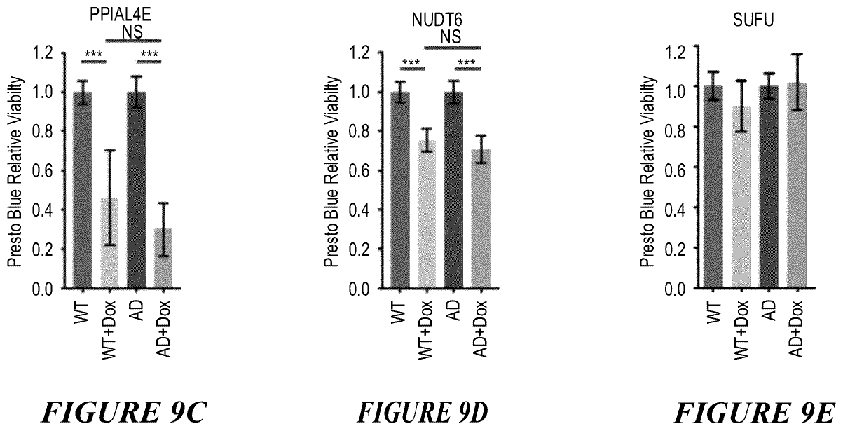

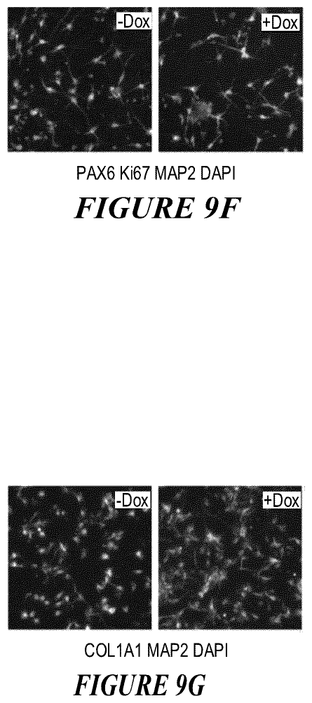

To perform secondary validation experiments and test many single guide RNAs simultaneously, the platform (FIG. 8A) was modified to transduce neural cells with guide RNAs at the end of the differentiation (DIV20). TOMM22, a previously described essential gene that was also essential in the screen, was used to confirm that transducing cells at Day 20 of the differentiation (FIG. 8C) gave similar results to transducing stem cells (FIG. 8B). To validate the WGS, the essential genes whose loss of function resulted in a significant loss of viability in both the wild-type and the APP^{swe/swe} neurons were the focus. As outlined in FIG. 9A, genes were selected for validation based on their combined beta score. The top 2 essential genes from the screen were PPIAL4E and NUDT6 FIG. 9B and CRISPR mediated knockout of either resulted in a significant loss of viability in neurons from both genotypes (FIG. 9C-9D).

The top ranked gene (SUFU) with a positive beta score (FIG. 9B) is involved in the SHH signaling pathway which has a documented role in both cell proliferation and neuronal survival. However, after 40 days in culture, no significant increase was observed in cell viability under the SUFU knockout condition FIG. 9E. Therefore, it was hypothesized that SUFU KO results in an increase in a rare (<1%) contaminant of proliferating cells remaining in the cultures at Day 30 (see FIG. 1D) and performed immunofluorescence experiments to address this. Immunofluorescence confirmed that Ki67+ cells were PAX6 negative (thus, not residual NPCs) but positive for the COL1A1, a marker of fibroblast-like cells including vascular leptomeningeal cells (VLMCs) recently described in hPSC-derived cultures. This indicates that SUFU knockout results in the increased proliferation of a very small population of non-neural cells in the culture (FIGS. 9F and 9G).

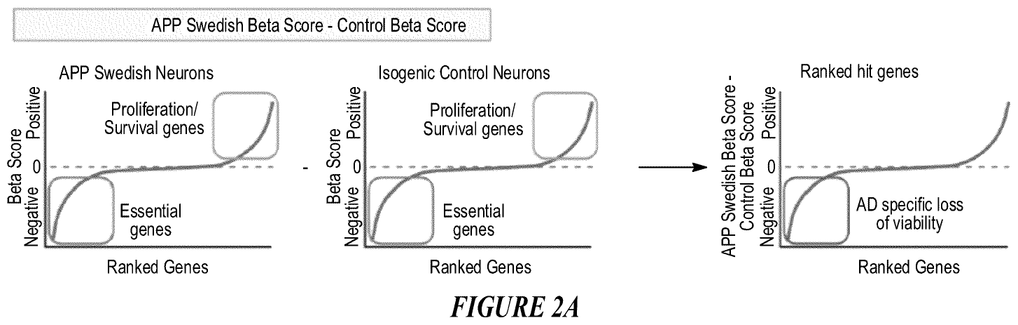

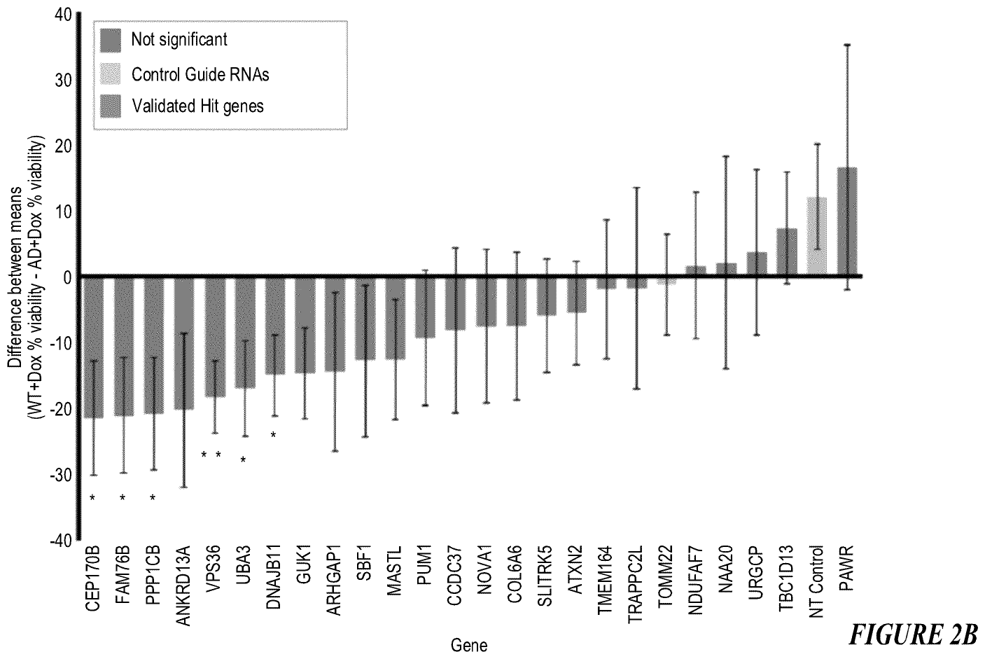

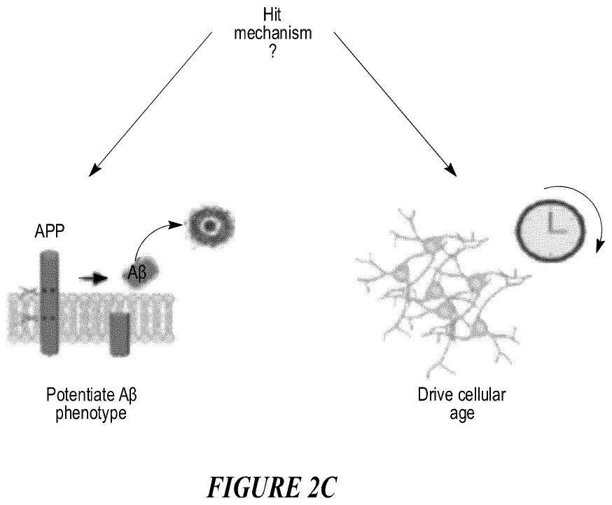

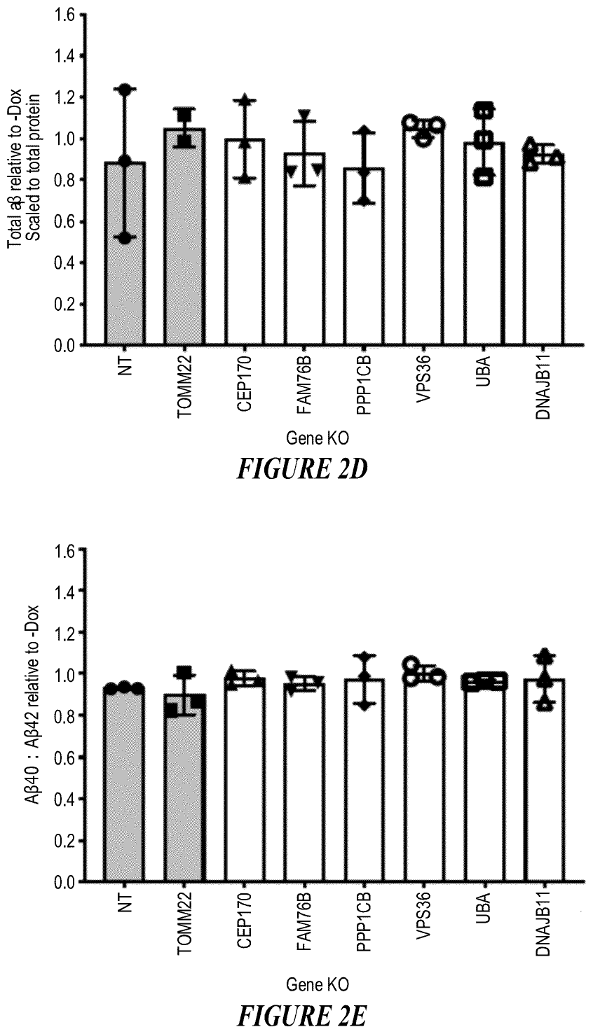

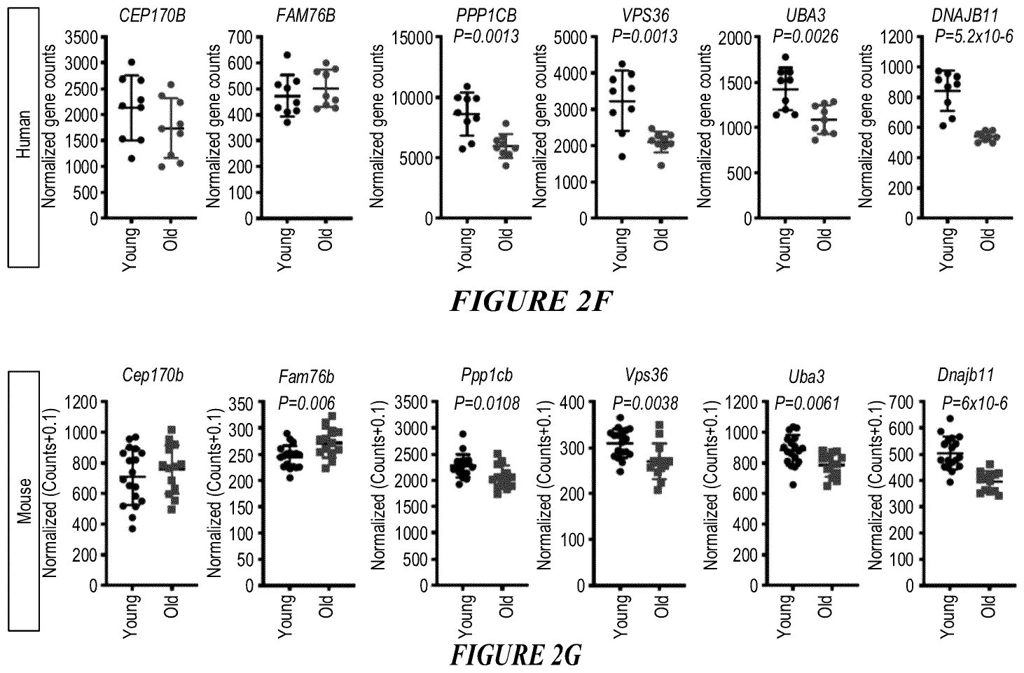

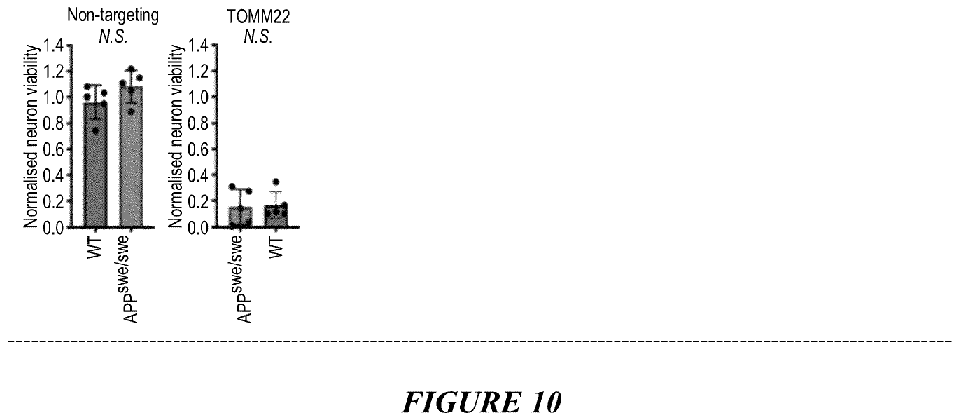

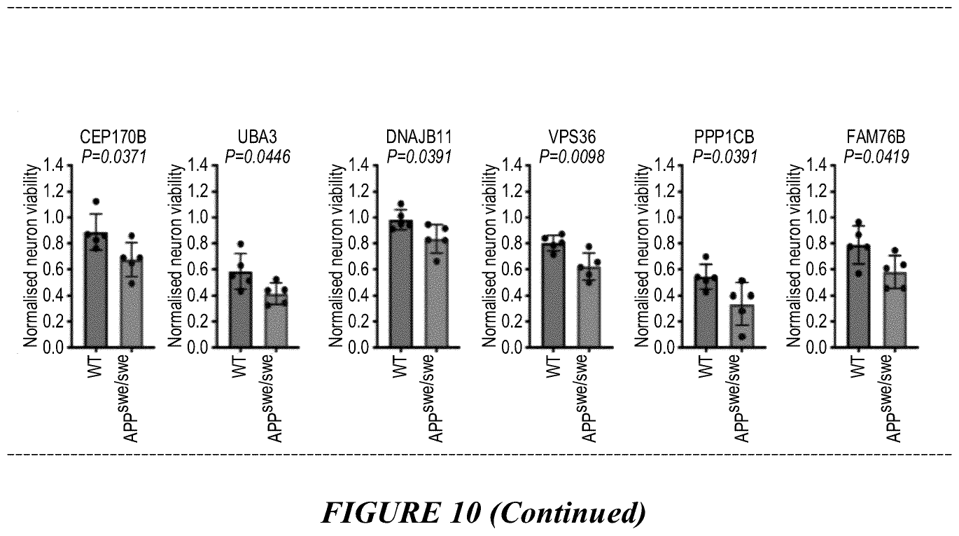

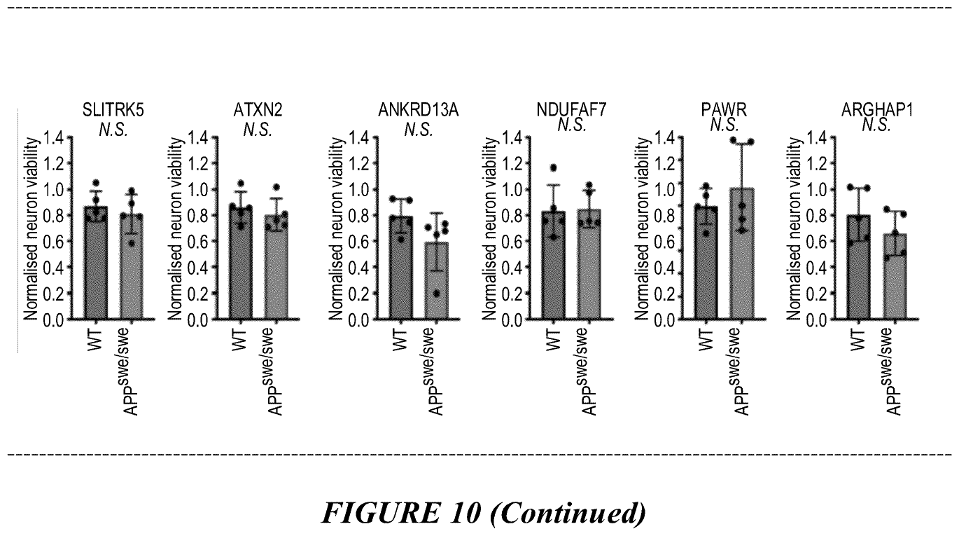

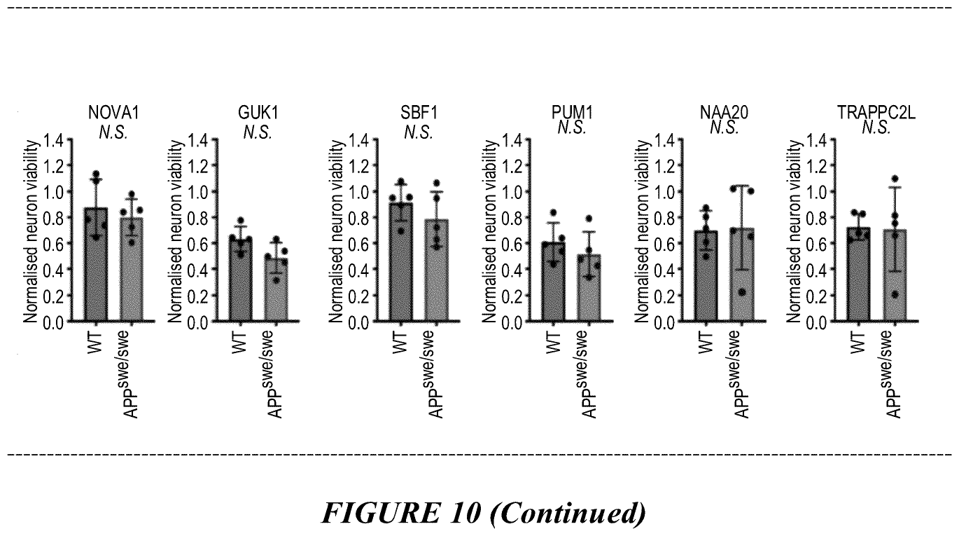

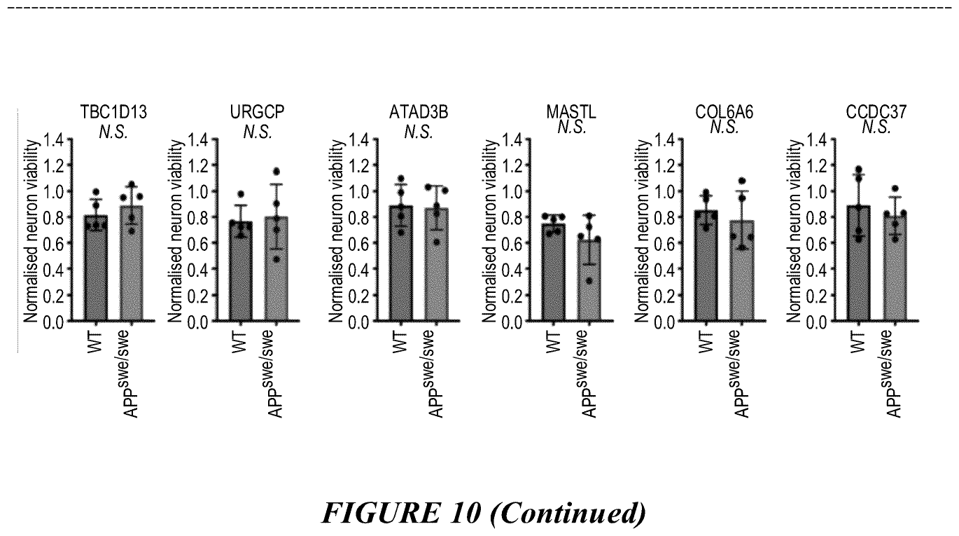

To select AD enhanced regulators of neuronal viability for further investigation, the list of genes that were significantly and specifically decreased in the AD neurons in both the T=0 control and endpoint control datasets were overlapped. This gave a total of 273 genes that were ranked according to the difference between the beta scores of the wild-type and APP^{swe/swe} neurons (FIG. 2A). In total, the top 24 genes were individually validated alongside a non-targeting guide (negative control) and TOMM22 (positive control). Of the genes tested, DNAJB11, CEP170B, FAM76B, PPP1CB, VPS36 and UBA3 showed a significant decrease in viability in APP^{swe/swe} neurons compared to wild-type neurons (FIG. 2B; FIG. 10).

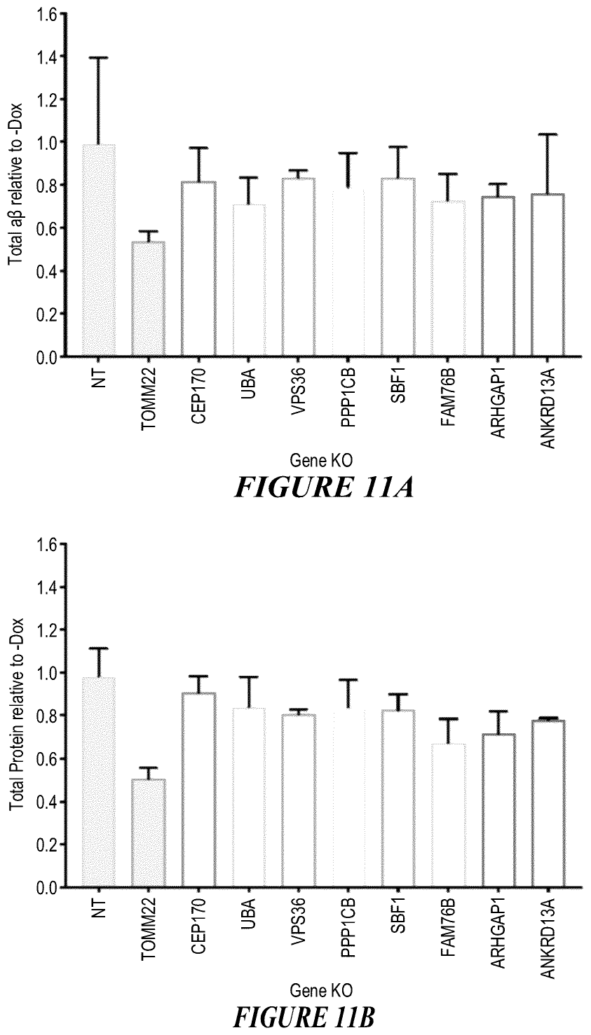

It was hypothesized that AD enhanced loss of viability seen in FIG. 2B could occur because of potentiation of the disordered APP processing seen in APP^{swe/swe} neurons or by triggering independent risk factors such as cellular age (FIG. 2C). To address this, electrochemical ELISA was performed to quantify Aβ peptide production. None of the hit genes following CRISPR-mediated loss of function triggered a change in the total amount of Aβ produced, as normalized to total protein levels (FIG. 2D; FIG. 11). There was also no change in the ratio of Aβ40 to the longer, more pathogenic Aβ42 (FIG. 2E). These results indicated that hit genes act independently of APP processing.

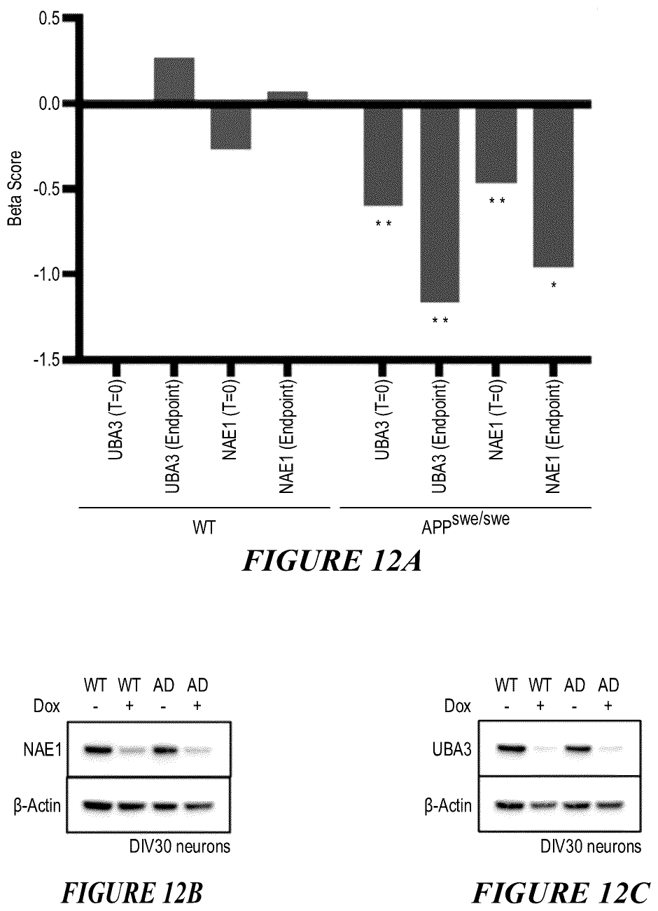

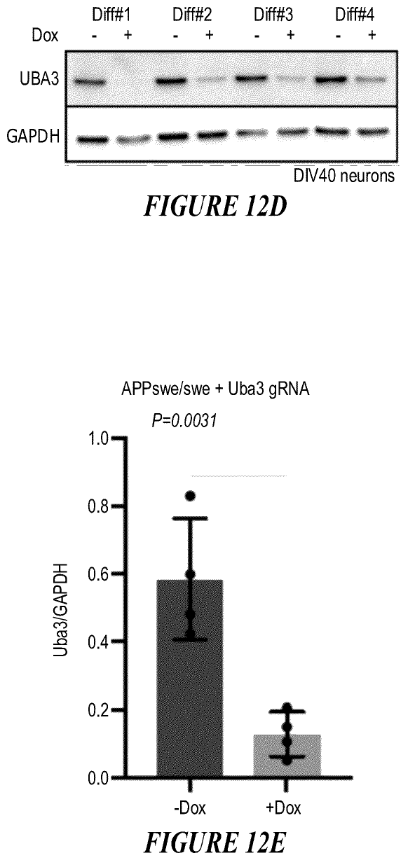

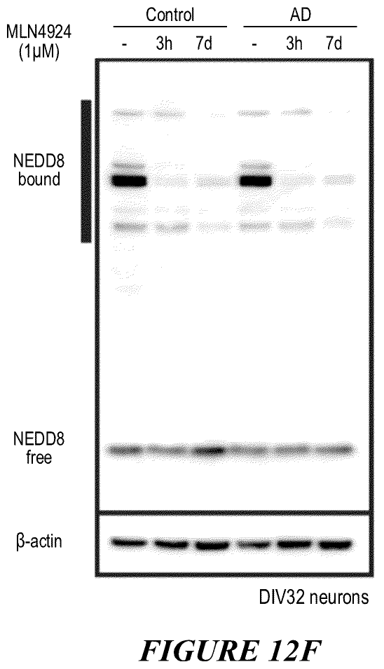

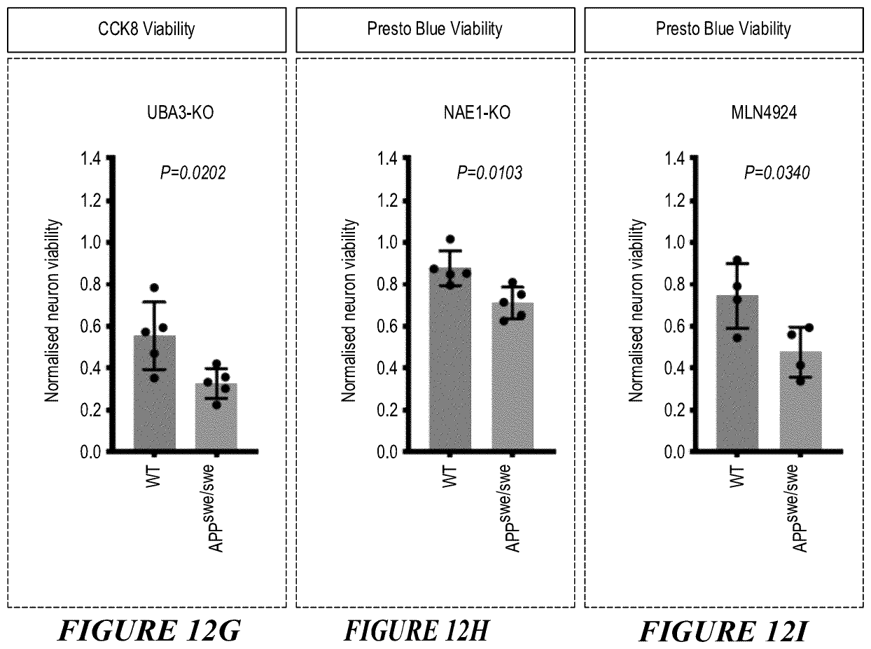

Existing RNA sequencing datasets were used to identify validated hit genes that are also significantly decreased in the aging human or mouse brain. It was postulated that genes that show age-dependent changes in expression are more likely to contribute to age-associated phenotypes. Of the 6 validated genes DNAJB11, UBA3, VPS36 and PPP1CB showed a significant decrease in both the aged mouse and human cortex. UBA3 was prioritized for further analysis because NAE1, the regulatory subunit of the Uba3-Nae1 E1 enzyme, was also a significant hit in the screen (FIG. 12A) providing additional validation that inhibiting neddylation has a more pronounced effect on the viability of APP^{swe/swe} versus isogenic control neurons. Interestingly, a neddylation inhibitor (MLN4924) is being tested as cancer therapeutic due to its ability to induce DNA damage and cellular senescence, phenotypes also associated with increased cellular age.

For further validation, western blots were performed to confirm that the guide RNAs used in this study resulted in a robust decrease in UBA3 or NAE1 protein in cortical neurons (FIG. 12B-12E). It was confirmed that 1 μM of MLN4924 was able to effectively block the conjugation of NEDD8 to target proteins in postmitotic cortical neurons (FIG. 12F). Then, additional assays were performed to confirm the loss of viability phenotype including repeating the secondary screen for UBA3 using an independent assay for cell viability (CCK8; formazan dye based; FIG. 12G). Consistent with UBA3 KO acting through its canonical function, the knockout of NAE1 or chemical inhibition of neddylation also resulted in a significantly more pronounced decrease in viability in the APP^{swe/swe} neurons (FIG. 12H-12I).

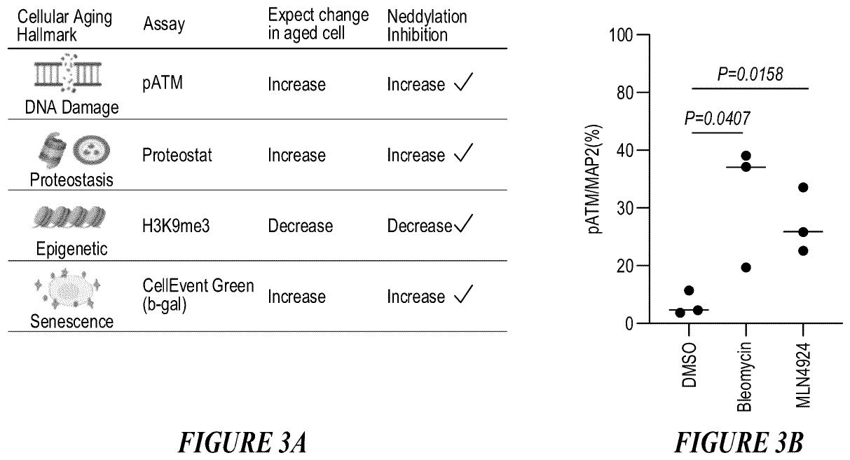

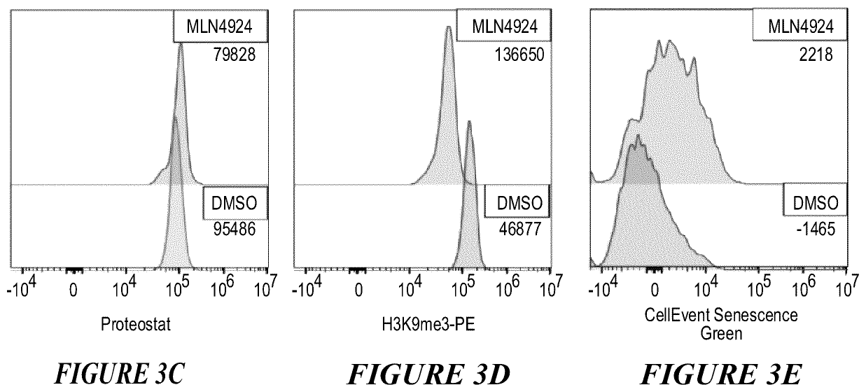

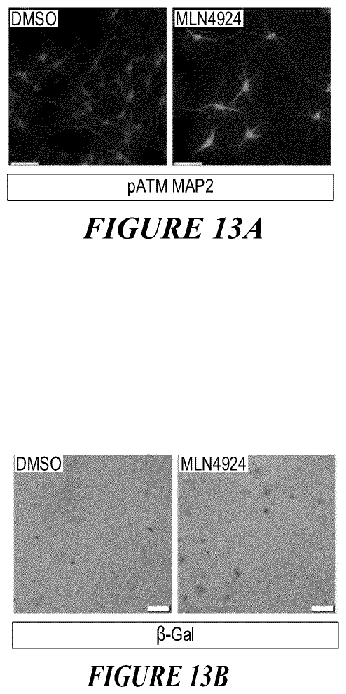

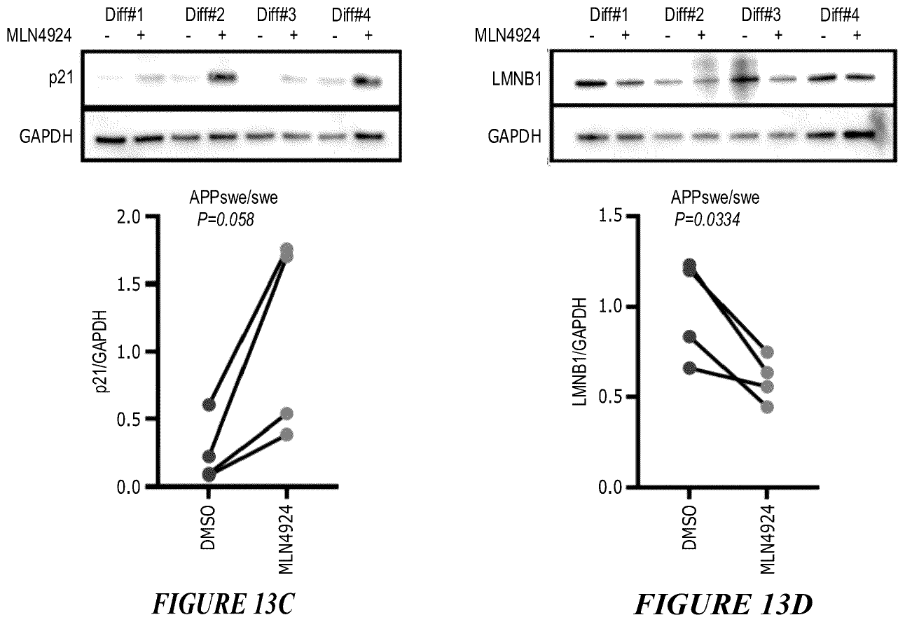

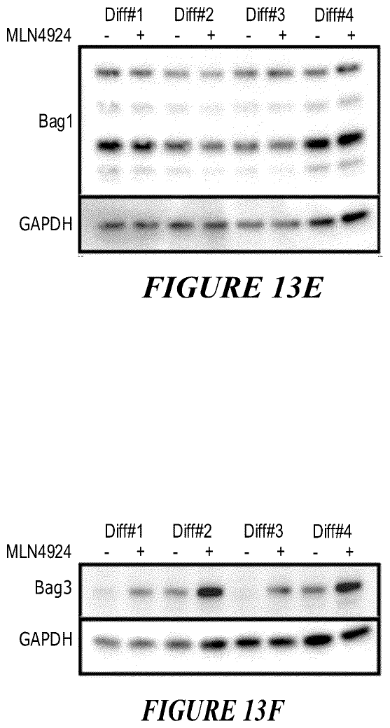

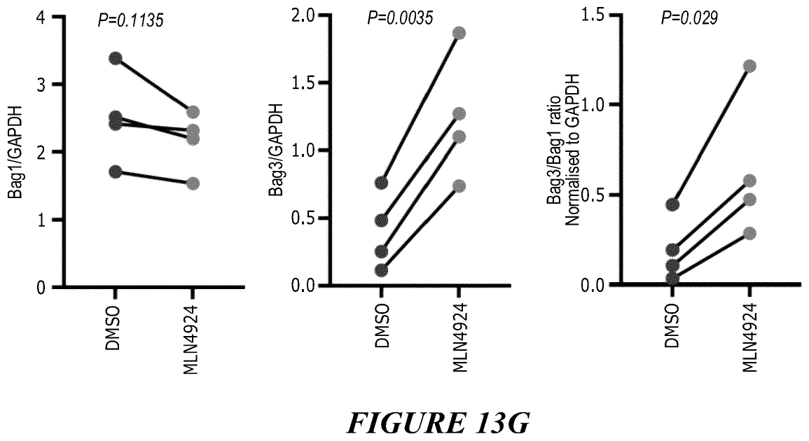

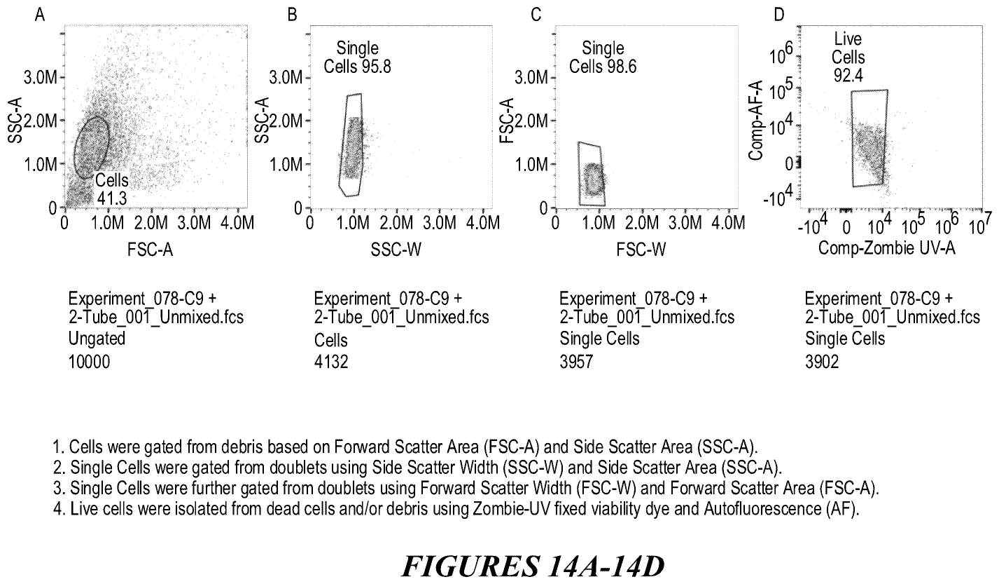

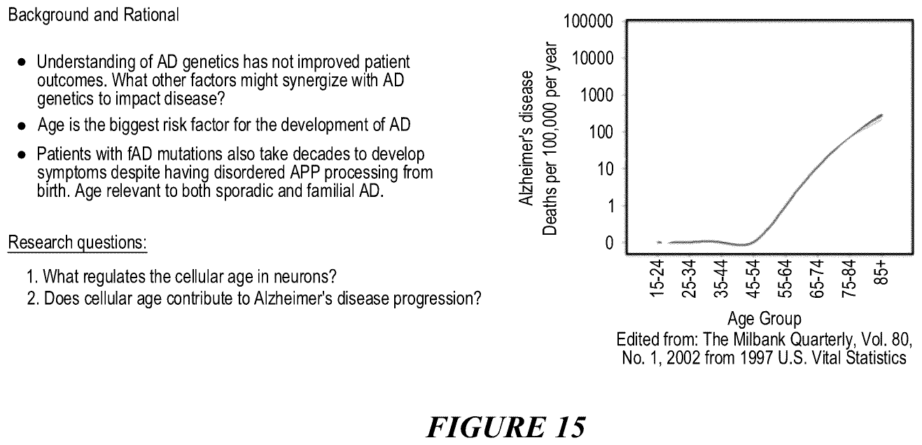

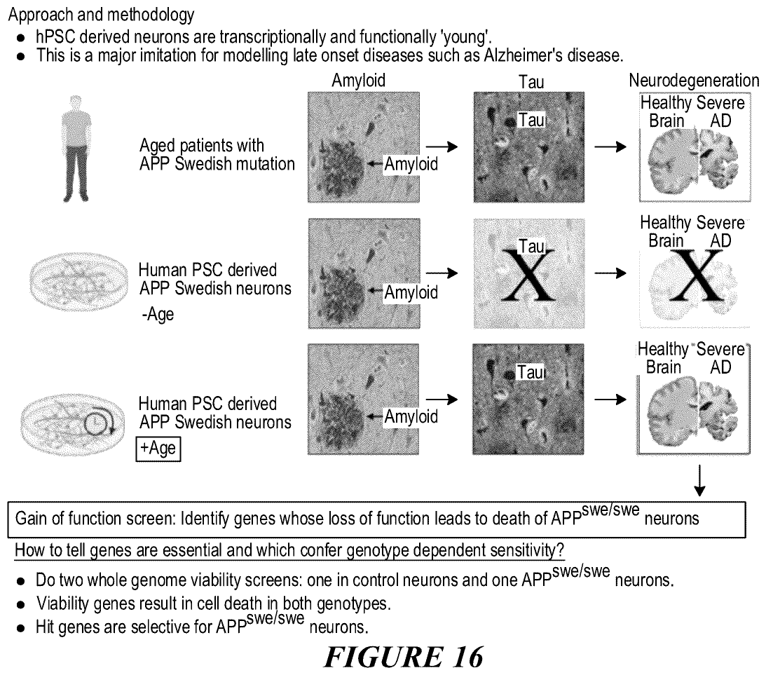

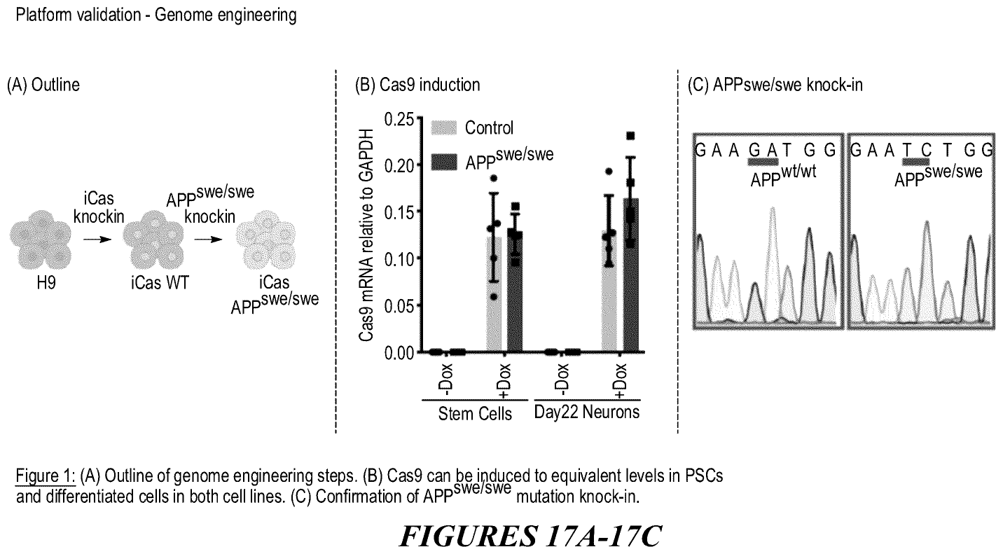

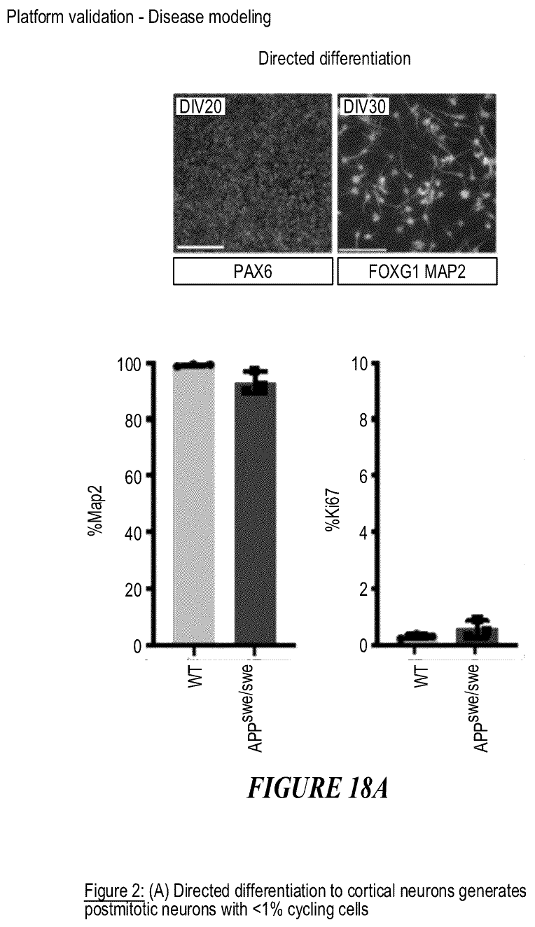

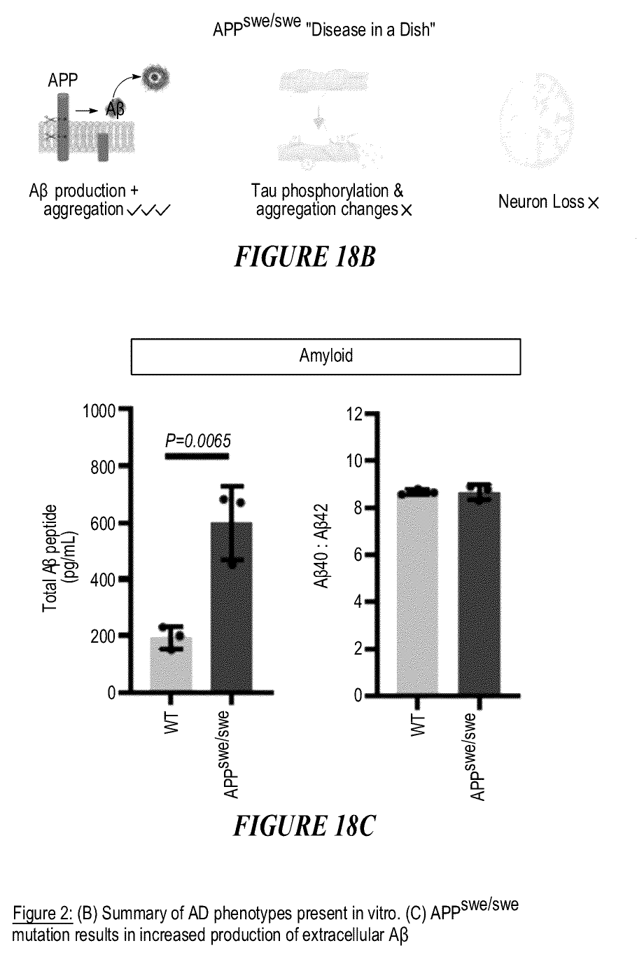

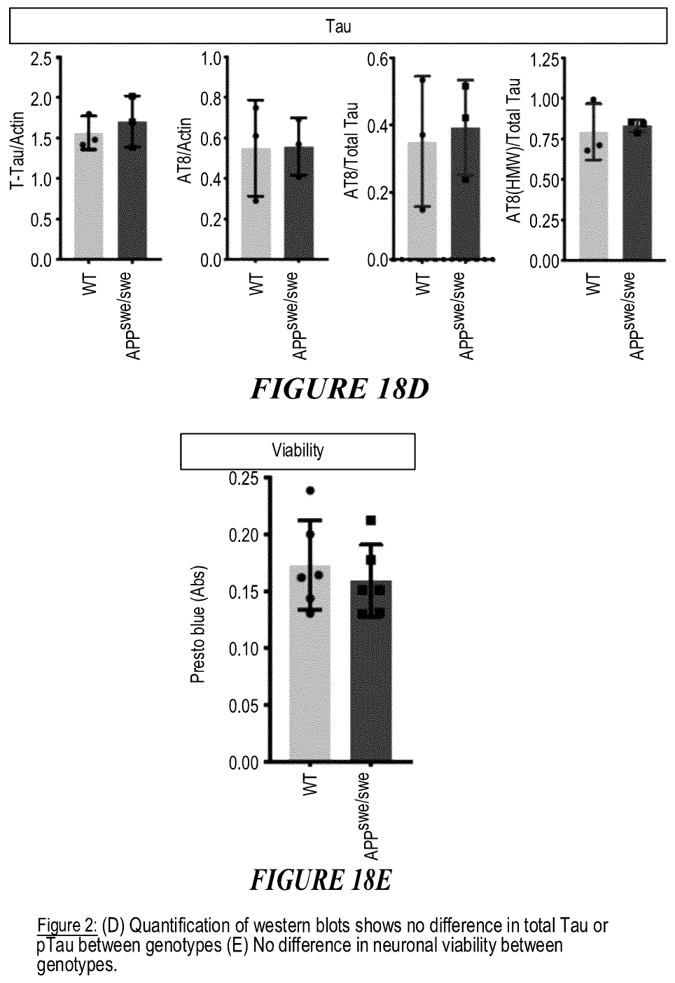

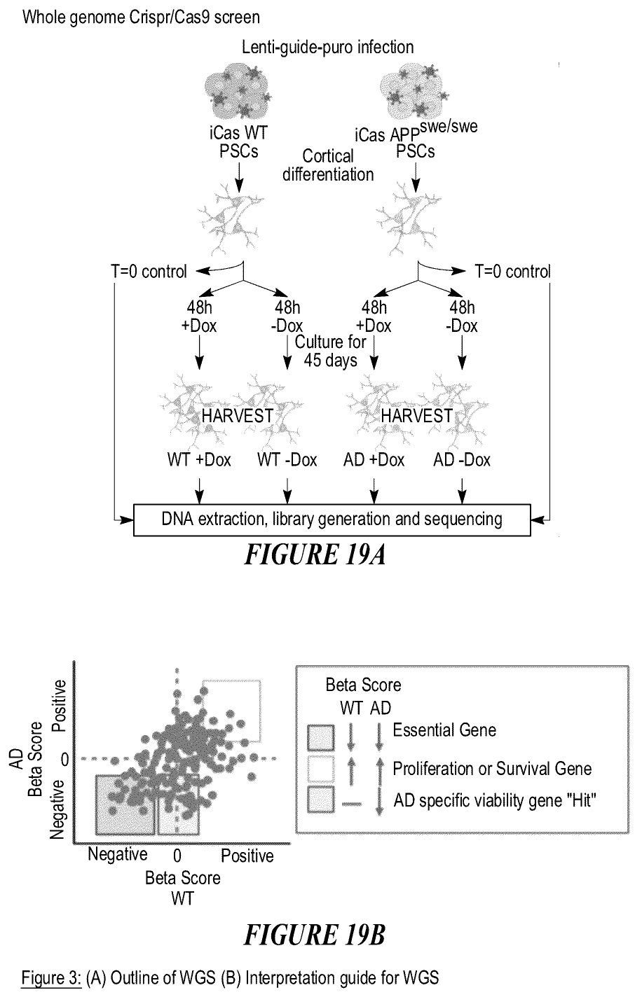

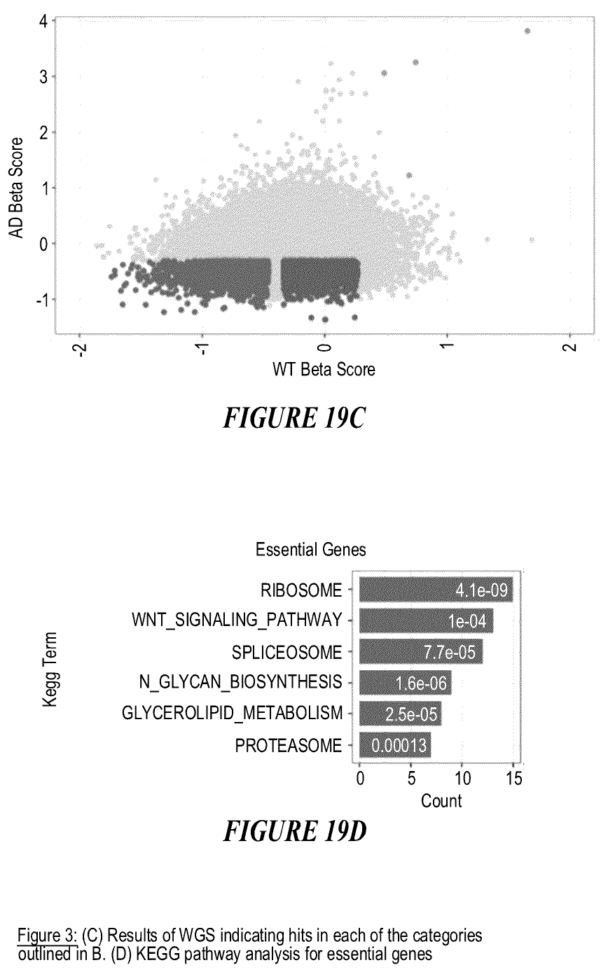

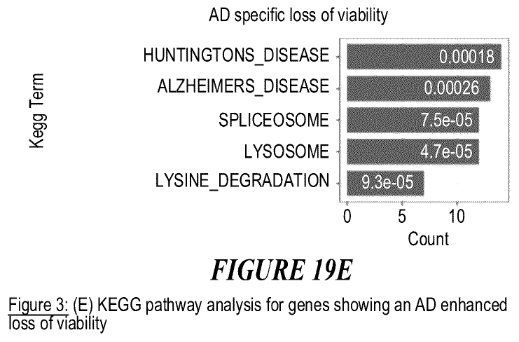

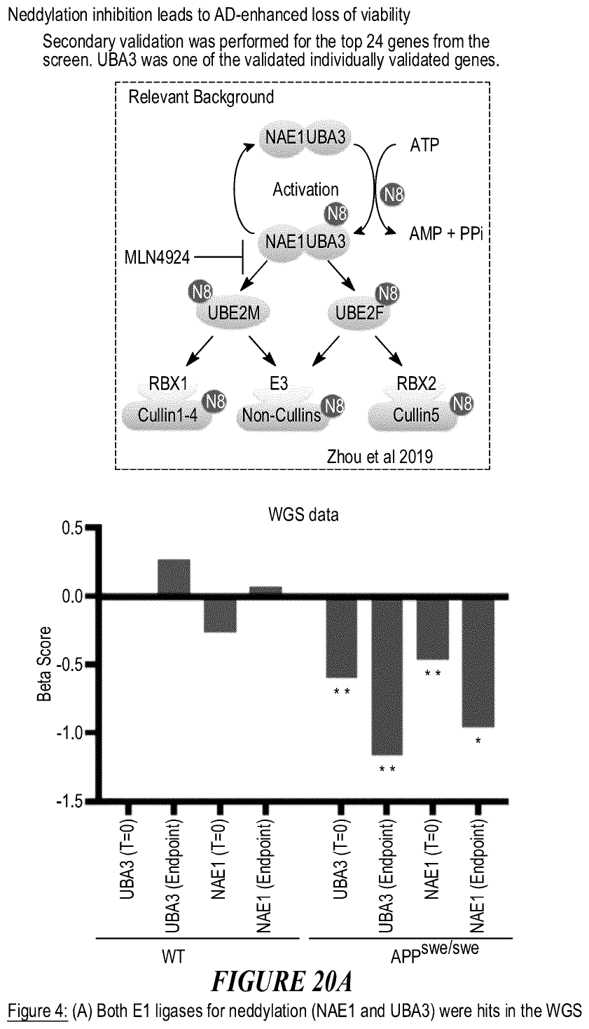

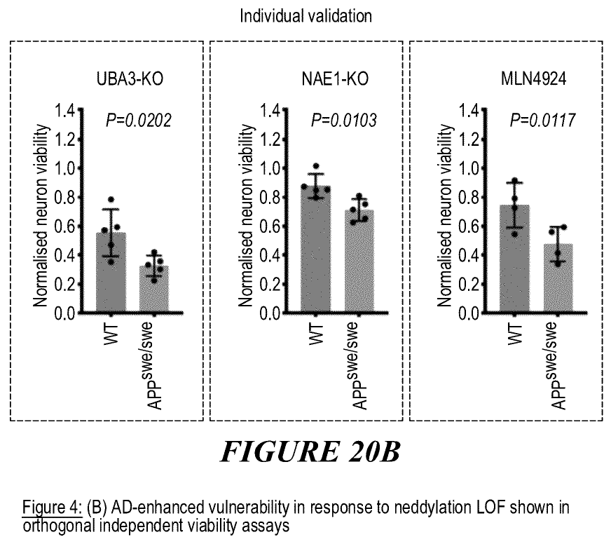

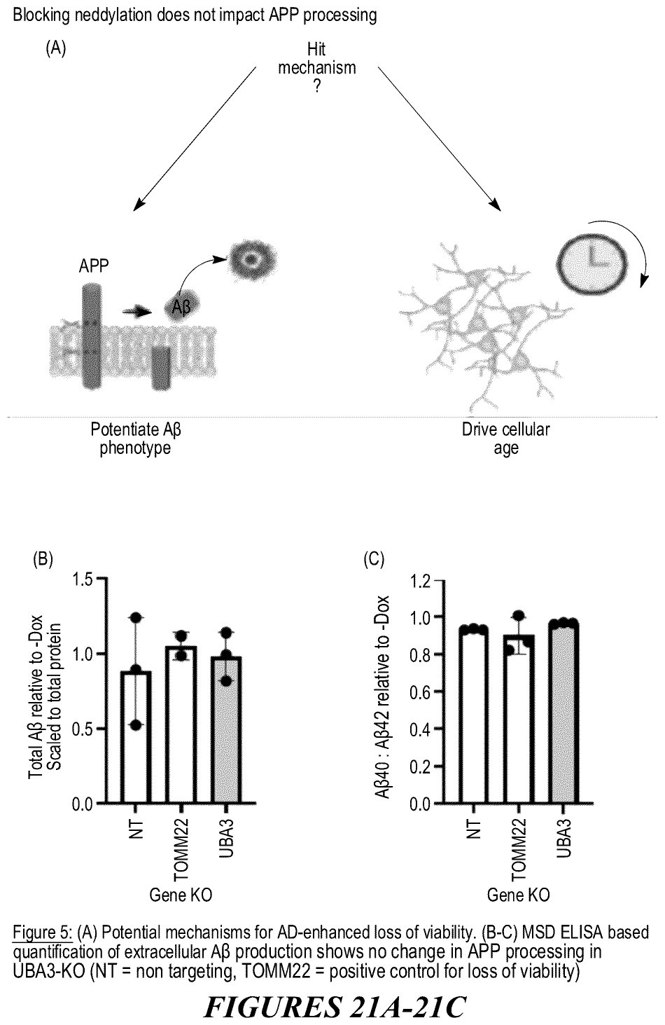

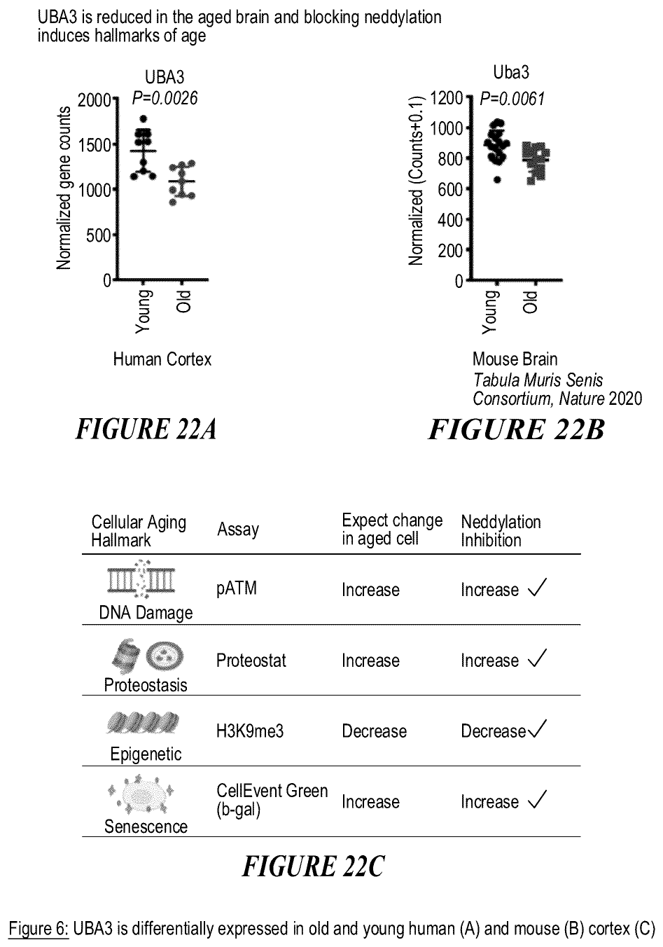

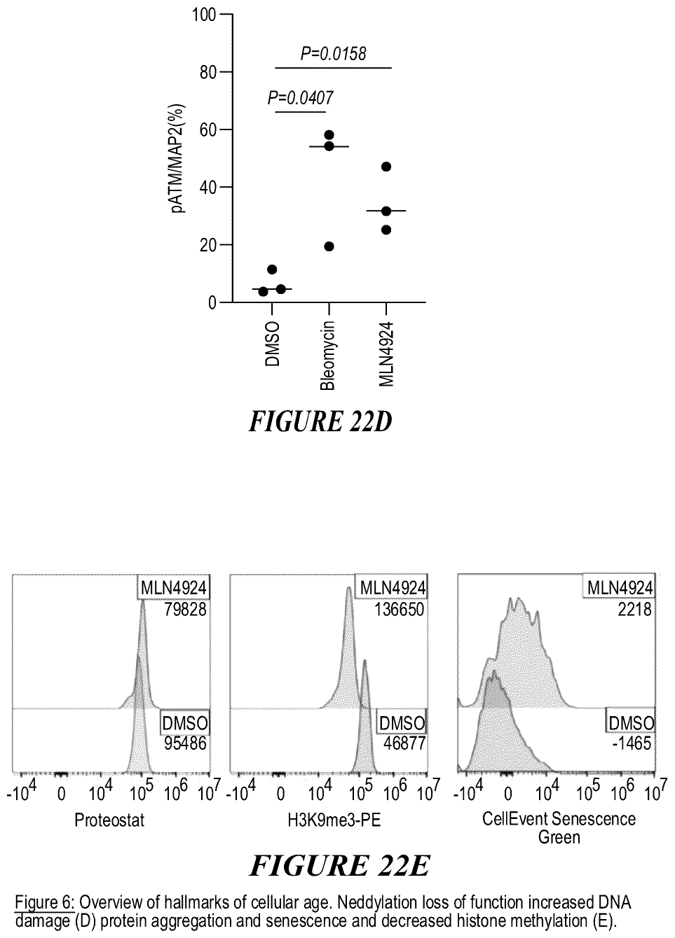

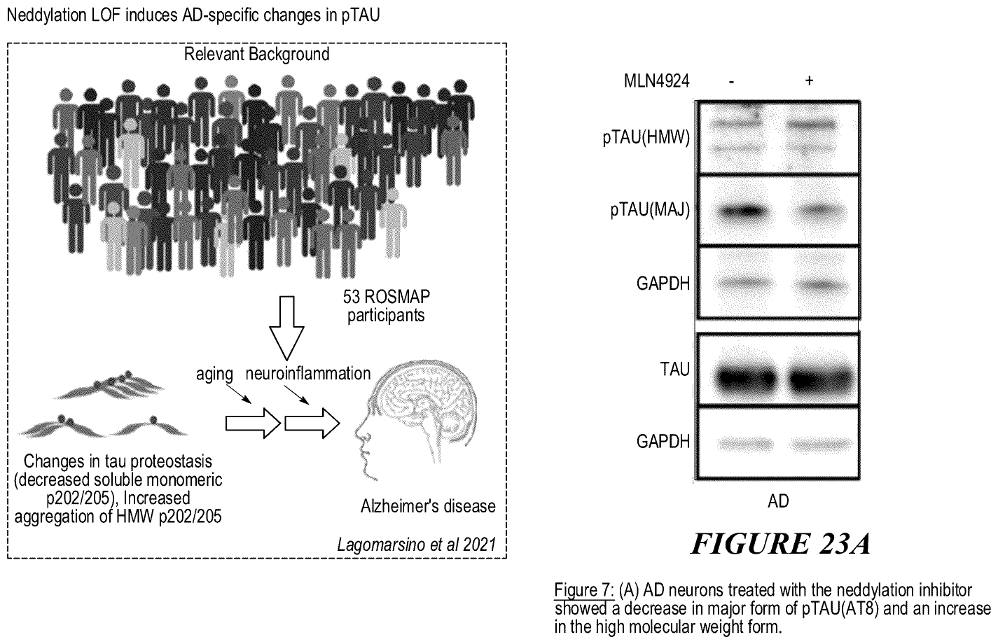

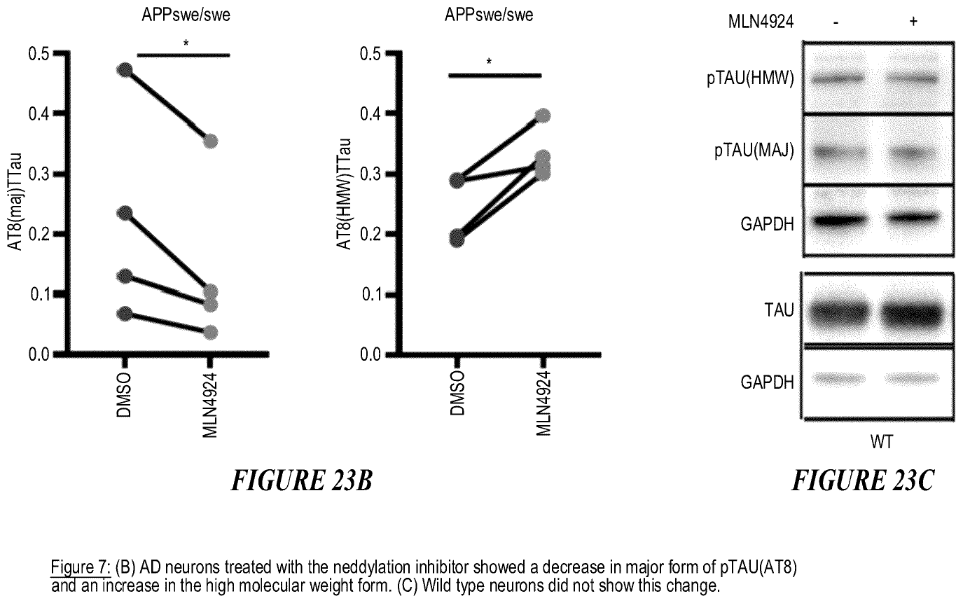

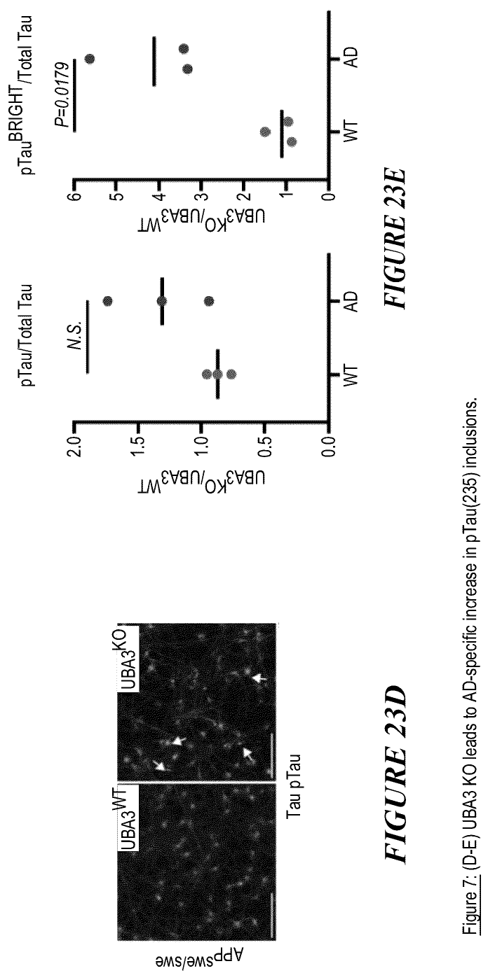

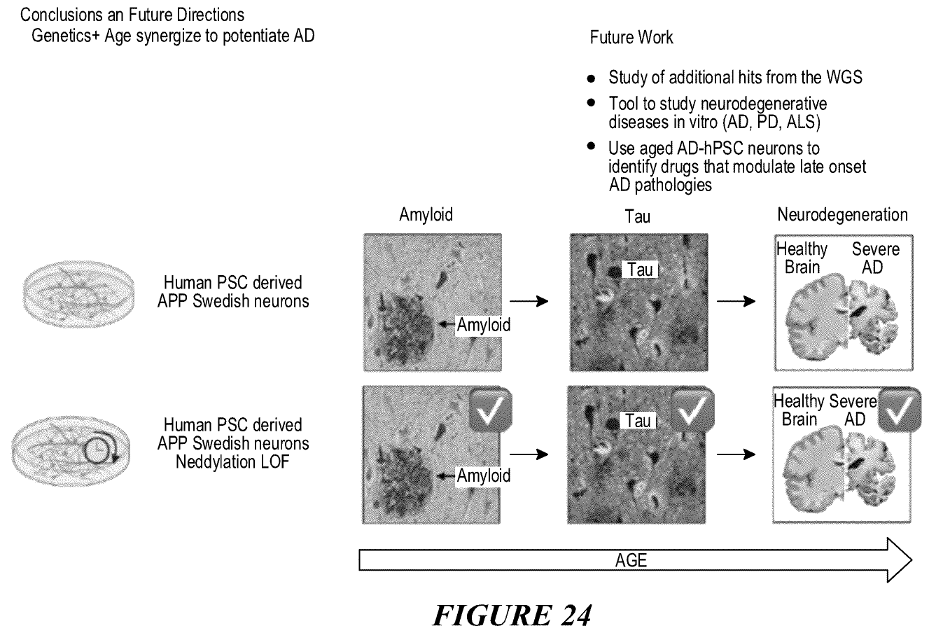

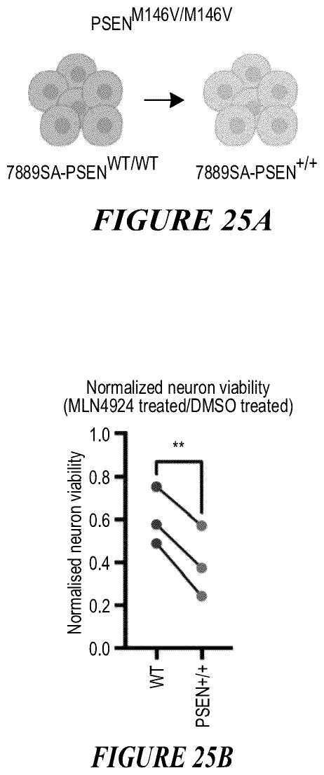

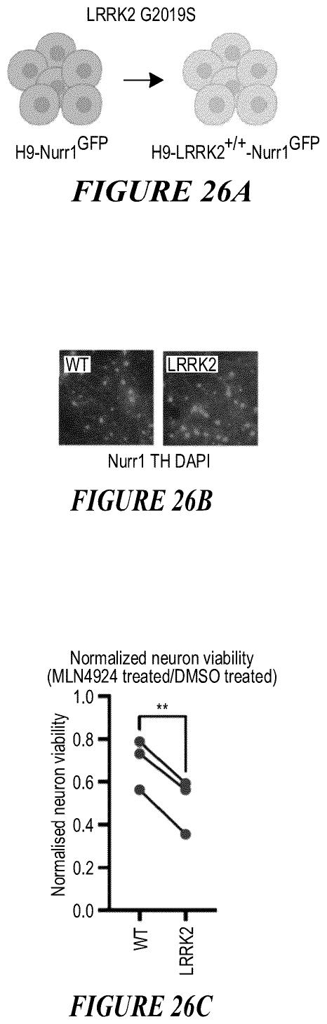

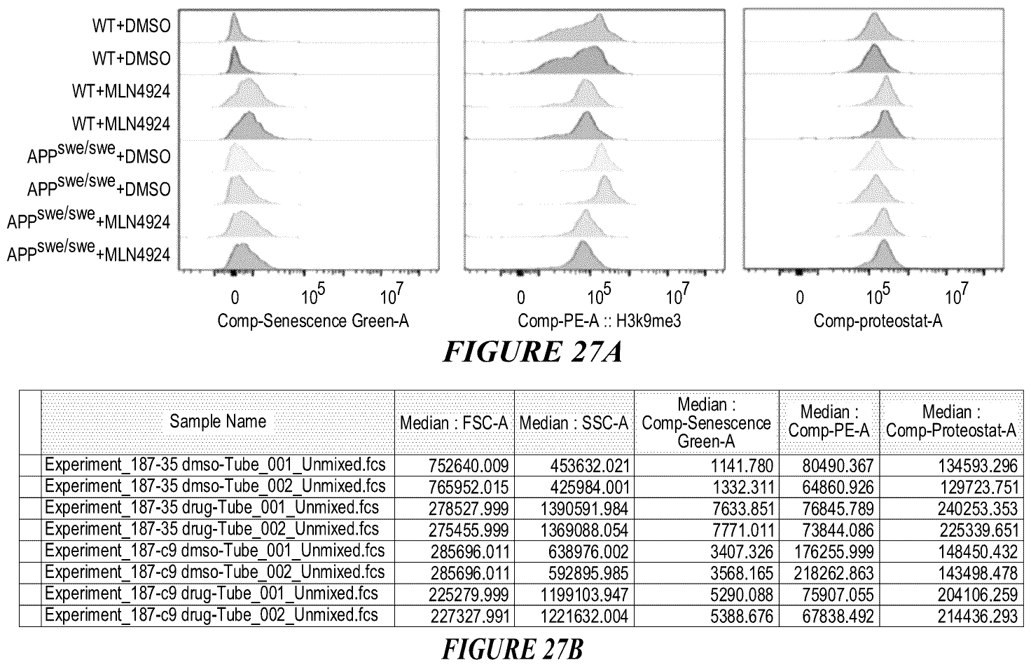

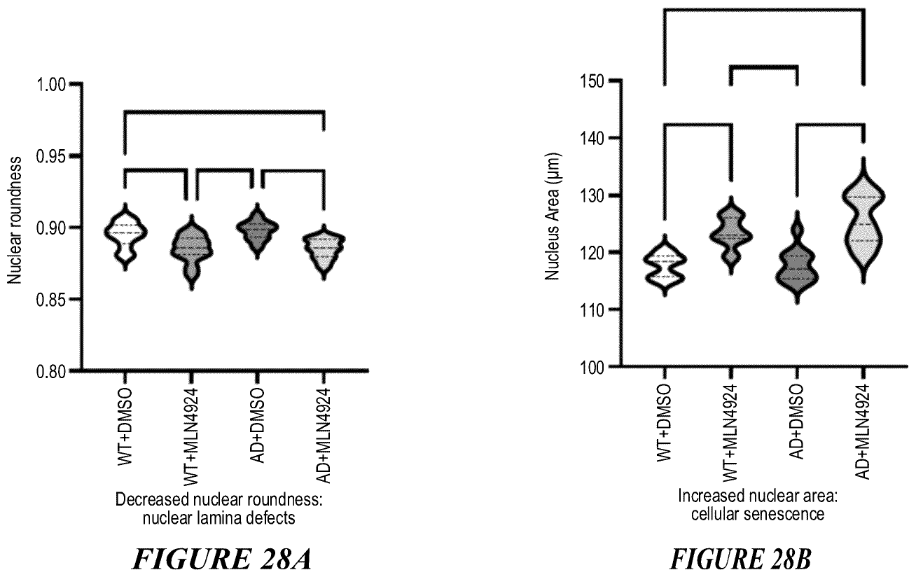

While UBA3 expression is decreased in the aged mouse and human brain (FIG. 2F), it is not known whether this decrease directly contributes to driving neuronal age. A set of cellular hallmarks of age have been established to be used as a readout of inducing age-like phenotypes or to measure cellular rejuvenation during reprogramming (FIG. 3A). Remarkably, inhibiting neddylation in AD cortical neurons induced multiple hallmarks of age (FIG. 3B and 3C) including an increase in cellular senescence, an increase in protein aggregation, loss of heterochromatin and increased DNA damage. DNA damage in neurons was assessed by activation of pATM (FIG. 3B, FIG. 13A) while several orthogonal assays were used to confirm induction of cellular senescence including senescence associated βGal expression by histochemistry, the induction of p21 and loss of LMNB 1 by Western blot (FIG. 13B-13D) Impaired proteostasis has been linked with both neurodegenerative disease and with cellular aging. For cellular aging, it has been reported that there is an increase in the BAG3/BAG1 ratio in the aged rodent brain and that this represents a switch from the UPS to the autophagic pathway for protein quality control. Inhibiting neddylation resulted in an increase in BAG3 relative to BAG1 protein by Western Blot mimicking age-related changes on proteostasis in cortical neurons (FIG. 13E-13G). Overall, these changes are all consistent with increased cellular age.

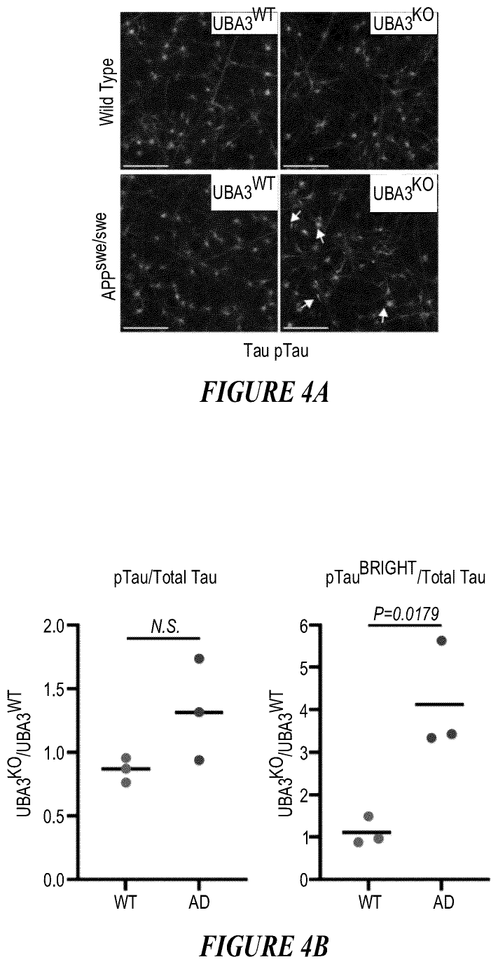

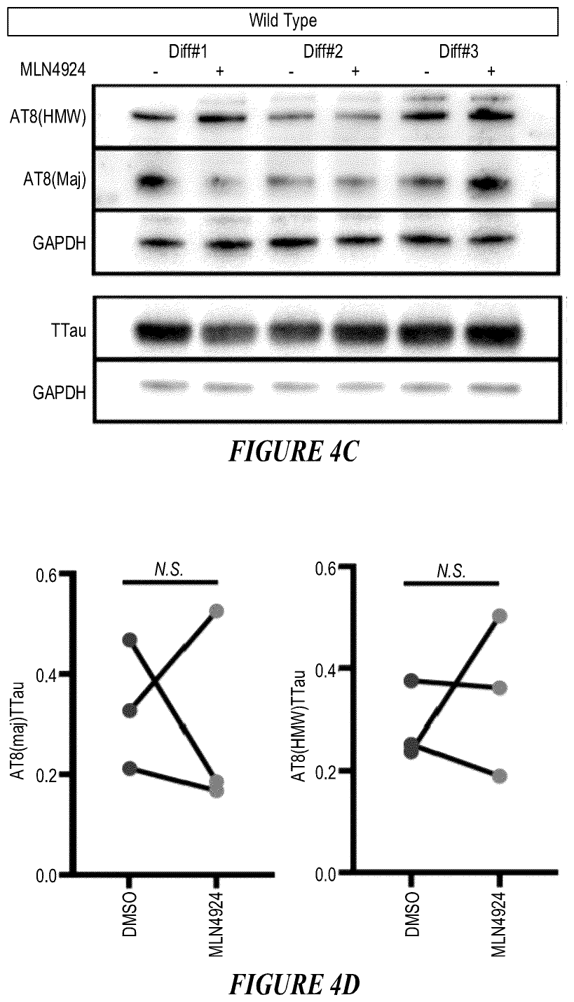

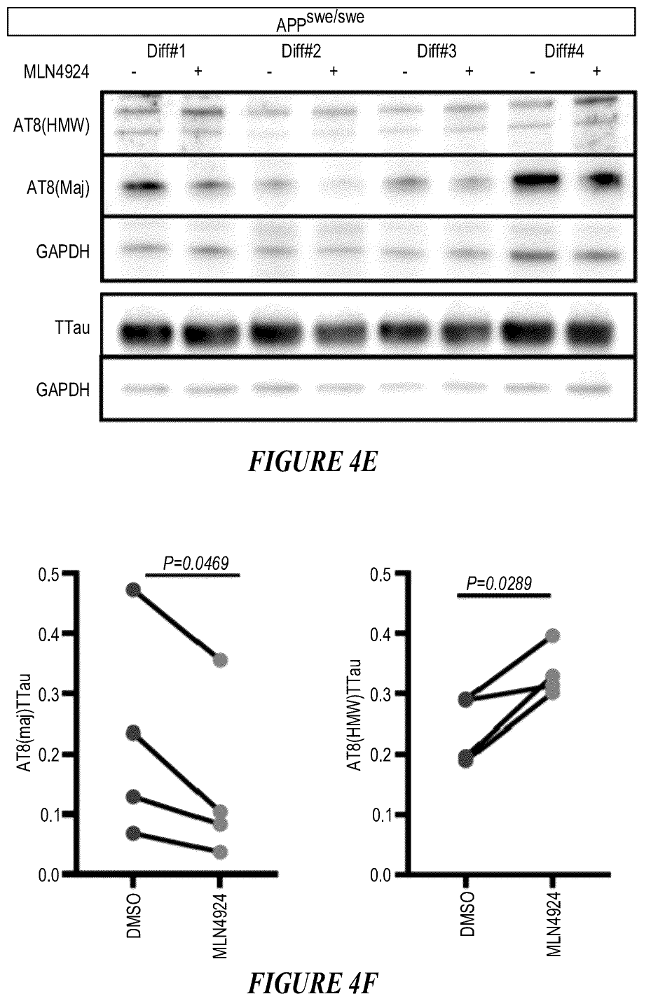

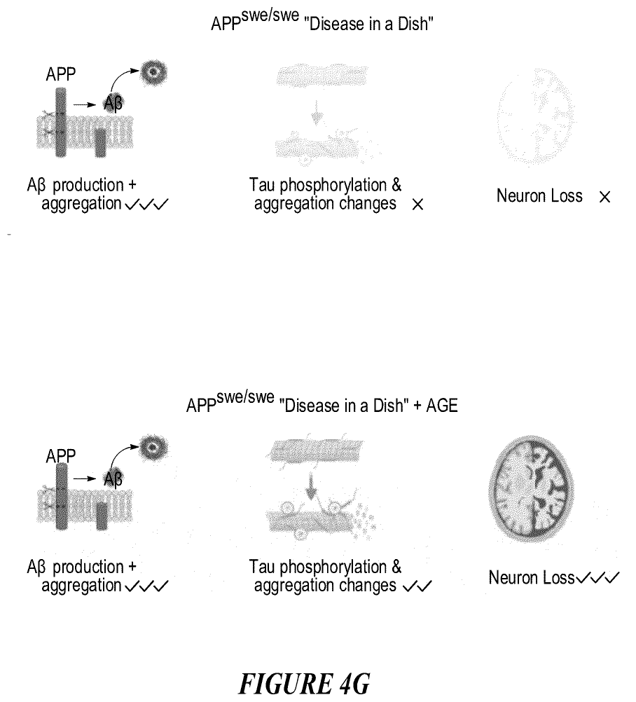

The instant data indicates that genetic or pharmacological inhibition of neddylation can trigger cortical neuron degeneration in an AD-dependent manner, thereby capturing a late disease phenotype not captured in a standard hPSC-based AD model. This raises the question of whether other late-stage Tau phenotypes can be captured in pharmacologically aged AD neurons. pTau(S235) was the focus of the instant analyses because it is one of the first sites of phosphorylation that can identify symptomatic AD. Immunofluorescence indicated that overall p-Tau(S235) was not significantly increased in AD cortical neurons lacking UBA3 relative to total Tau levels. However, there was a significant increase in p-Tau(S235)+inclusions in the UBA3 KO APP^{swe/swe} neurons (FIG. 4A and 4B). Brightly stained inclusions could be identified in both the cell body and in neurites (white and yellow arrows respectively FIG. 4A). Finally, these inclusions were specific to App^{swe/swe} neurons and rarely detected in wild-type neurons.

Whether inhibiting neddylation can drive Tau-related changes that are characteristic not only to early disease progression such as p-Tau(S235) but also to later stages of disease progression such as p-Tau(S202/T205) was tested. pTau(T205) occurs later in disease progression and is associated with fibril formation. In particular, high molecular weight (HMW; >250 kDA) oligomeric hyperphosphorylated p-Tau (S202/T205) has been shown to promote the seeding of Tau aggregation. Finally, in iPSC derived neurons, decreased pTau (MAJ; 50 kDA) and increased HMW tau have been shown to correlate with cognitive decline and the presence of tangles in the postmortem brain. Western blot analysis for p-Tau (S202/T205) showed that inhibiting neddylation had no impact on levels of either the major(MAJ) or HMW forms of p-Tau (S202/T205) in wild-type cortical neurons (FIG. 4C and 4D). In contrast, inhibiting neddylation in APP^{swe/swe} neurons resulted in a reduction in ratio of p-Tau(MAJ) to Total Tau and a significant increase in the ratio of p-Tau(HMW) to Total Tau (FIG. 4E and 4F). In summary, the instant data suggest that increased Aβ peptide production in APP^{swe/swe} neurons combined with impaired neddylation can induce Tau pathology in addition to triggering AD-related cortical neuron degeneration. Accordingly, inhibiting neddylation in APP^{swe/swe} neurons can capture all the three major AD related disease phenotypes: abnormal Aβ production, disease-related Tau phosphorylation and aggregation phenotypes and direct AD-related cortical neuron loss (FIG. 4G).

### Discussion

Using a whole genome CRISPR screening approach, neddylation was identified as a regulator of neuronal aging and it was shown that cellular aging can synergize with AD-genetics to trigger late onset AD phenotypes in vitro. These findings indicate that cellular aging can have a causal impact on the progression of fAD and highlight the importance of developing therapeutic strategies to reverse cellular aging. In addition to the confirmation of the neddylation pathway in this study, several other genes were identified and validated whose loss of function had a more pronounced impact on the viability of APP^{swe/swe} neurons than control neurons. Further characterization of hit genes may define a more complete set of genes and pathways capable of potentiating fAD disease. For example, two of the validated hit genes VPS36 and PPP1CB have also been linked to Tau propagation and phosphorylation respectively.

This study further has several implications for in vitro disease modelling efforts. A question is whether capturing age-related features in neurons will reveal late-stage phenotypes in other familial or sporadic AD-iPSC models or trigger age-related features and disease phenotypes in other neuronal lineages and late-onset neurodegenerative disorders such as Parkinson's disease or ALS. Finally, parallel efforts focus on the identification of strategies that drive neuronal maturation independent of neuronal age. It is conceivable such efforts to accelerate maturation could be combined with the presently disclosed induced aging platform to better capture synaptic or spine degeneration, phenotypes that for human neurons are currently limited largely to in vivo and postmortem studies. In conclusion, protein neddylation is identified as a physiologically relevant regulator of neuronal age and increased cellular age can contribute to the potentiation of AD phenotypes in in vitro human PSC models of disease.

Although the presently disclosed subject matter and its advantages have been described in detail, it should be understood that various changes, substitutions and alterations can be made herein without departing from the spirit and scope of the present disclosure. Moreover, the scope of the present application is not intended to be limited to the particular embodiments of the process, machine, manufacture, and compositions of matter, means, methods and steps described in the specification. As one of ordinary skill in the art will readily appreciate from the present disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps. Various patents, patent applications, publications, product descriptions, protocols, and sequence accession numbers are cited throughout this application, this present disclosures of which are incorporated herein by reference in their entireties for all purposes.

What is claimed is:

1. A method of preparing an in vitro model of neurodegenerative disease comprising modulating protein neddylation in a population of neurons; wherein the population of neurons exhibit genetic mutation of at least one gene that is associated with neurodegenerative disease.

2. The method of claim 1, wherein modulating protein neddylation comprises exposing the population of neurons to a compound that modulates protein neddylation.

3. The method of claim 2, wherein the at least one compound that modulates protein neddylation is selected from the group consisting of MLN4924, TAS4464, CSN5i-3, ZM223, NACM-OPT, Keap1-Nrf2-IN-4, WS-383, VII-31, derivatives thereof, and combinations thereof.

4. The method of claim 1, wherein modulating protein neddylation comprises modifying expression of at least one gene which regulates protein neddylation pathways.

5. The method of claim 4, wherein the at least one gene which regulates protein neddylation pathways is selected from the group consisting of UBA3, NAE1, and combinations thereof.

6. The method of claim 1, wherein the mutation of at least one gene that is associated with neurodegenerative disease results in increased production of amyloid-beta peptide.

7. The method of claim 1, wherein the mutation of at least one gene that is associated with neurodegenerative disease results in a change in the ratio of Aβ40 to Aβ42 peptide.

8. The method of claim 1, wherein the at least one gene that is associated with neurodegenerative disease comprises the Amyloid-beta precursor protein (APP) gene.

9. The method of claim 8, wherein the mutation of the APP gene comprises K595N/M596L.

10. The method of claim 1, wherein the at least one gene that is associated with neurodegenerative disease comprises the presenilin-1 (PSEN) gene.

11. The method of claim 10, wherein the mutation of the PSEN gene comprises M146V.

12. The method of claim 1, wherein the mutation of at least one gene that is associated with neurodegenerative disease results in increased production of N-ethylmaleimide sensitive factor (NSF) aggregates.

13. The method of claim 1, wherein the at least one gene that is associated with neurodegenerative disease comprises the (LRRK2) gene.

14. The method of claim 13, wherein the mutation of the LRRK2 gene comprises G2019S.

15. The method of claim 1, wherein the neurodegenerative disease is Alzheimer's disease, Parkinson's disease, Amyotrophic Lateral Sclerosis (ALS), or Huntington's disease.

16. The method of claim 1, wherein the neurons are obtained from in vitro differentiation of stem cells.

17. The method of claim 16, wherein the stem cells are human stem cells selected from the group consisting of human embryonic stem cells, human induced pluripotent stem cells, human parthenogenetic stem cells, human primordial germ cell-like pluripotent stem cells, human epiblast stem cells, human F-class pluripotent stem cells, and combinations thereof.

18. The method of claim 1, wherein the neurons are cortical neurons.

19. A method of identifying genes associated with cellular aging and/or progression of neurodegenerative disease comprising:
a) obtaining a first population of neurons;
b) obtaining a second population of neurons, and modifying expression of a test gene in the second population of neurons;
c) measuring functional activity of the second population of neurons relative to the first population of neurons;
wherein the first population of neurons and the second population of neurons exhibit genetic mutation of at least one gene that is associated with neurodegenerative disease;
wherein a difference in the functional activity between the first population of neurons and the second population of neurons indicates that the test gene is associated with cellular aging and/or progression of neurodegenerative disease.

20. A composition for identifying genes associated with cellular aging and/or progression of neurodegenerative disease comprising a population of neurons, wherein the population of neurons exhibit genetic mutation at a test gene, wherein the population of neurons exhibit genetic mutation of at least one gene that is associated with neurodegenerative disease.


SEQUENCE LISTING (42 sequences)

SEQ ID NO: 1 - DNA, 20 bp, synthetic construct
  source 1..20: mol_type = other DNA
  misc_feature 1..20: note = Description of sequence: Cas9 Fwd primer
ccgaagaggtcgtgaagaag

SEQ ID NO: 2 - DNA, 20 bp, synthetic construct
  source 1..20: mol_type = other DNA
  misc_feature 1..20: note = Description of sequence: Cas9 Rev primer
gccttatccagttcgctcag

SEQ ID NO: 3 - DNA, 20 bp, synthetic construct
  source 1..20: mol_type = other DNA
  misc_feature 1..20: note = Description of sequence: FOXG1 Fwd primer
caacggcatctacgagttca

SEQ ID NO: 4 - DNA, 20 bp, synthetic construct
  source 1..20: mol_type = other DNA
  misc_feature 1..20: note = Description of sequence: FOXG1 Rev primer
tgttgagggacagattgtgg

SEQ ID NO: 5 - DNA, 20 bp, synthetic construct
  source 1..20: mol_type = other DNA
  misc_feature 1..20: note = Description of sequence: GAPDH Fwd primer
atgttcgtcatgggtgtgaa

SEQ ID NO: 6 - DNA, 20 bp, synthetic construct
  source 1..20: mol_type = other DNA
  misc_feature 1..20: note = Description of sequence: GAPDH Rev primer
aggggtgctaagcagttggt

SEQ ID NO: 7 - DNA, 20 bp, synthetic construct
  source 1..20: mol_type = other DNA
  misc_feature 1..20: note = Description of sequence: OCT4 Fwd primer
ctgggttgatcctcggacct

SEQ ID NO: 8 - DNA, 19 bp, synthetic construct
  source 1..19: mol_type = other DNA
  misc_feature 1..19: note = Description of sequence: OCT4 Rev primer
ccatcggagttgctctcca

SEQ ID NO: 9 - DNA, 20 bp, synthetic construct
  source 1..20: mol_type = other DNA
  misc_feature 1..20: note = Description of sequence: PAX6 Fwd primer
gtccatctttgcttgggaaa

SEQ ID NO: 10 - DNA, 20 bp, synthetic construct
  source 1..20: mol_type = other DNA
  misc_feature 1..20: note = Description of sequence: PAX6 Rev primer
tagccaggttgcgaagaact

SEQ ID NO: 11 - DNA, 18 bp, synthetic construct
  source 1..18: mol_type = other DNA
  misc_feature 1..18: note = Description of sequence: TUBB3 Fwd primer
cgatgccatgctcatcac

SEQ ID NO: 12 - DNA, 20 bp, synthetic construct
  source 1..20: mol_type = other DNA
  misc_feature 1..20: note = Description of sequence: TUBB3 Rev primer
cccagtatgagggagatcgt

SEQ ID NO: 13 - RNA, 20 nt, synthetic construct
  source 1..20: mol_type = other RNA
  misc_feature 1..20: note = Description of sequence: Non-targeting sgRNA
agcgcagatagcgcgtatca

SEQ ID NO: 14 - RNA, 20 nt, synthetic construct
  source 1..20: mol_type = other RNA
  misc_feature 1..20: note = Description of sequence: TOMM22 sgRNA
acagctagatgagaccctgt

SEQ ID NO: 15 - RNA, 20 nt, synthetic construct
  source 1..20: mol_type = other RNA
  misc_feature 1..20: note = Description of sequence: SUFU sgRNA
tggcccgcagagttaatgca

SEQ ID NO: 16 - RNA, 20 nt, synthetic construct
  source 1..20: mol_type = other RNA
  misc_feature 1..20: note = Description of sequence: PPIAL4E sgRNA
ccagggtttatgtgtcaggg

SEQ ID NO: 17 - RNA, 20 nt, synthetic construct
  source 1..20: mol_type = other RNA
  misc_feature 1..20: note = Description of sequence: NUDT6 sgRNA
ggatatgcttcacatcaagt

SEQ ID NO: 18 - RNA, 20 nt, synthetic construct
  source 1..20: mol_type = other RNA
  misc_feature 1..20: note = Description of sequence: NAE1 sgRNA
tcaaagaagcagtatcggca

SEQ ID NO: 19 - RNA, 20 nt, synthetic construct
  source 1..20: mol_type = other RNA
  misc_feature 1..20: note = Description of sequence: CEP170B sgRNA
ggaacacacaccatactgcg

SEQ ID NO: 20 - RNA, 20 nt, synthetic construct
  source 1..20: mol_type = other RNA
  misc_feature 1..20: note = Description of sequence: UBA3 sgRNA
cattccaggcagaatcaccc

SEQ ID NO: 21 - RNA, 20 nt, synthetic construct
  source 1..20: mol_type = other RNA
  misc_feature 1..20: note = Description of sequence: DNAJB11 sgRNA
atcttatagaaatctcgtct

SEQ ID NO: 22 - RNA, 20 nt, synthetic construct
  source 1..20: mol_type = other RNA
  misc_feature 1..20: note = Description of sequence: VPS36 sgRNA
tcagtcggtgtgtactaaga

SEQ ID NO: 23 - RNA, 20 nt, synthetic construct
  source 1..20: mol_type = other RNA
  misc_feature 1..20: note = Description of sequence: PPP1CB sgRNA
gatccagataaggatgtgca

SEQ ID NO: 24 - RNA, 20 nt, synthetic construct
  source 1..20: mol_type = other RNA
  misc_feature 1..20: note = Description of sequence: FAM76B sgRNA
cctttcgaggagctctccca

SEQ ID NO: 25 - RNA, 20 nt, synthetic construct
  source 1..20: mol_type = other RNA
  misc_feature 1..20: note = Description of sequence: SLITRK5 sgRNA
cgacatgcgctccattaagt

SEQ ID NO: 26 - RNA, 20 nt, synthetic construct
  source 1..20: mol_type = other RNA
  misc_feature 1..20: note = Description of sequence: ATXN2 sgRNA
aatagagaagtcatatcctg

SEQ ID NO: 27 - RNA, 20 nt, synthetic construct
  source 1..20: mol_type = other RNA
  misc_feature 1..20: note = Description of sequence: ANKRD13A sgRNA
ggaaaggtcgaagcgttcgg

SEQ ID NO: 28 - RNA, 20 nt, synthetic construct
  source 1..20: mol_type = other RNA
  misc_feature 1..20: note = Description of sequence: NDUFAF7 sgRNA
gacacctttcatatacactg

SEQ ID NO: 29 - RNA, 20 nt, synthetic construct
  source 1..20: mol_type = other RNA
  misc_feature 1..20: note = Description of sequence: PAWR sgRNA
agtacgaagatgatgaagca

SEQ ID NO: 30 - RNA, 20 nt, synthetic construct
  source 1..20: mol_type = other RNA
  misc_feature 1..20: note = Description of sequence: ARHGAP1 sgRNA
cttgcggtcaaactcccggt

SEQ ID NO: 31 - RNA, 20 nt, synthetic construct
  source 1..20: mol_type = other RNA
  misc_feature 1..20: note = Description of sequence: NOVA1 sgRNA
gatgcgatctggattaacgg

SEQ ID NO: 32 - RNA, 20 nt, synthetic construct
  source 1..20: mol_type = other RNA
  misc_feature 1..20: note = Description of sequence: GUK1 sgRNA
ggcatgctcgatgaagtcgc

SEQ ID NO: 33 - RNA, 20 nt, synthetic construct
  source 1..20: mol_type = other RNA
  misc_feature 1..20: note = Description of sequence: SBF1 sgRNA
ttgaccggagaaatgcggaa

SEQ ID NO: 34 - RNA, 20 nt, synthetic construct
  source 1..20: mol_type = other RNA
  misc_feature 1..20: note = Description of sequence: PUM1 sgRNA
tcttagacaggaatcgcccg

SEQ ID NO: 35 - RNA, 20 nt, synthetic construct
  source 1..20: mol_type = other RNA
  misc_feature 1..20: note = Description of sequence: NAA20 sgRNA
accaaggcgtcgaaattctg

SEQ ID NO: 36 - RNA, 20 nt, synthetic construct
  source 1..20: mol_type = other RNA
  misc_feature 1..20: note = Description of sequence: TRAPPC2L sgRNA
ggtgcacacatctctggacg

SEQ ID NO: 37 - RNA, 20 nt, synthetic construct
  source 1..20: mol_type = other RNA
  misc_feature 1..20: note = Description of sequence: TBC1D13 sgRNA
gtggcaaaggtgtagtagag

SEQ ID NO: 38 - RNA, 20 nt, synthetic construct
  source 1..20: mol_type = other RNA
  misc_feature 1..20: note = Description of sequence: URGCP sgRNA
ccgagtcaggcaacacgagt

SEQ ID NO: 39 - RNA, 20 nt, synthetic construct
  source 1..20: mol_type = other RNA
  misc_feature 1..20: note = Description of sequence: ATAD3B sgRNA
ccggccggtctcccaaaccg

SEQ ID NO: 40 - RNA, 20 nt, synthetic construct
  source 1..20: mol_type = other RNA
  misc_feature 1..20: note = Description of sequence: MASTL sgRNA
gaaggtgtgggattgactac

SEQ ID NO: 41 - RNA, 20 nt, synthetic construct
  source 1..20: mol_type = other RNA
  misc_feature 1..20: note = Description of sequence: COL6A6 sgRNA
gtttagcgatacctatcacc

SEQ ID NO: 42 - RNA, 20 nt, synthetic construct
  source 1..20: mol_type = other RNA
  misc_feature 1..20: note = Description of sequence: CCDC37 sgRNA
cagtgttaactccacaccag